SEQUENCE LISTING (41 sequences)

SEQ ID NO: 1 - protein, 12 aa, Artificial Sequence
  P1 mimotope
HSWTNSWMATFL

SEQ ID NO: 2 - protein, 9 aa, Artificial Sequence
  P2 cyclic mimotope
CNNSNMPLC

SEQ ID NO: 3 - protein, 14 aa, Artificial Sequence
  P2 cyclic mimotope with linker
ACNNSNMPLCGGGS

SEQ ID NO: 4 - protein, 14 aa, Artificial Sequence
  Scrambled control peptide
ACSPNLNMNCGGGS

SEQ ID NO: 5 - protein, 12 aa, Artificial Sequence
  Consensus mimotope sequence
XXXXXXXXXXXX

SEQ ID NO: 6 - protein, 12 aa, Artificial Sequence
  Peptide mimotope
NSWTNASLSTFH

SEQ ID NO: 7 - protein, 12 aa, Artificial Sequence
  Peptide mimotope
NSRTNNSQWTFQ

SEQ ID NO: 8 - protein, 12 aa, Artificial Sequence
  Peptide mimotope
ESWTNSWAHYFG

SEQ ID NO: 9 - protein, 12 aa, Artificial Sequence
  Peptide mimotope
ESWTNSWAMYFG

SEQ ID NO: 10 - protein, 12 aa, Artificial Sequence
  Peptide mimotope
QSYTNDDVLRIS

SEQ ID NO: 11 - protein, 12 aa, Artificial Sequence
  Peptide mimotope
QNMNNWTLASIM

SEQ ID NO: 12 - protein, 12 aa, Artificial Sequence
  Peptide mimotope
EVMNNWTLASIM

SEQ ID NO: 13 - protein, 12 aa, Artificial Sequence
  Peptide mimotope
ASISNLTLSRFM

SEQ ID NO: 14 - protein, 12 aa, Artificial Sequence
  Peptide mimotope
HSWSNYWGHQHA

SEQ ID NO: 15 - protein, 12 aa, Artificial Sequence
  Peptide mimotope
HRISNYAMELHS

SEQ ID NO: 16 - protein, 12 aa, Artificial Sequence
  Peptide mimotope
HSLTNTQMTQLS

SEQ ID NO: 17 - protein, 12 aa, Artificial Sequence
  Peptide mimotope
HSLSNIQMATLA

SEQ ID NO: 18 - protein, 12 aa, Artificial Sequence
  Peptide mimotope
HRMTNAMHHFMG

SEQ ID NO: 19 - protein, 12 aa, Artificial Sequence
  Peptide mimotope
HRMTNNAMDVFM

SEQ ID NO: 20 - protein, 12 aa, Artificial Sequence
  Peptide mimotope
HRLTNSEQAALP

SEQ ID NO: 21 - protein, 12 aa, Artificial Sequence
  Peptide mimotope
TAVTNSMMERLW

SEQ ID NO: 22 - protein, 12 aa, Artificial Sequence
  Peptide mimotope
GWGNKTPSQDVH

SEQ ID NO: 23 - protein, 12 aa, Artificial Sequence
  Peptide mimotope
DYTNSVSMRYLS

SEQ ID NO: 24 - protein, 12 aa, Artificial Sequence
  Peptide mimotope
HQLSNKDEQTPQ

SEQ ID NO: 25 - protein, 12 aa, Artificial Sequence
  Peptide mimotope
ADPFSPTNRIPL

SEQ ID NO: 26 - protein, 7 aa, Artificial Sequence
  Consensus cyclic mimotope sequence
XXXXXXX

SEQ ID NO: 27 - protein, 7 aa, Artificial Sequence
  Peptide mimotope
NNWNMPL

SEQ ID NO: 28 - protein, 7 aa, Artificial Sequence
  Peptide mimotope
NNRNMPL

SEQ ID NO: 29 - protein, 7 aa, Artificial Sequence
  Peptide mimotope
NNYNMPL

SEQ ID NO: 30 - protein, 7 aa, Artificial Sequence
  Peptide mimotope
NNQNMPL

SEQ ID NO: 31 - protein, 7 aa, Artificial Sequence
  Peptide mimotope
NNWKMPL

SEQ ID NO: 32 - protein, 7 aa, Artificial Sequence
  Peptide mimotope
NNSHMPL

SEQ ID NO: 33 - protein, 7 aa, Artificial Sequence
  Peptide mimotope
KNSXQPL

SEQ ID NO: 34 - protein, 7 aa, Artificial Sequence
  Peptide mimotope
NNSXMPL

SEQ ID NO: 35 - protein, 7 aa, Artificial Sequence
  Peptide mimotope
QNSHMPL

SEQ ID NO: 36 - protein, 7 aa, Artificial Sequence
  Peptide mimotope
NNSKMRL

SEQ ID NO: 37 - protein, 7 aa, Artificial Sequence
  Peptide mimotope
DWAPHFT

SEQ ID NO: 38 - protein, 12 aa, Artificial Sequence
  Control peptide
IMGVGAVGAGAI

SEQ ID NO: 39 - protein, 4 aa, Artificial Sequence
  peptide spacer
GGGS

SEQ ID NO: 40 - protein, 4 aa, Artificial Sequence
  Peptide spacer
GGGC

SEQ ID NO: 41 - protein, 4 aa, Artificial Sequence
  Peptide spacer
GGGK

(19) United States
(12) Patent Application Publication
(10) Pub. No.: US 2015/0056209 A1
(43) Pub. Date: Feb. 26, 2015

(54) PEPTIDE MIMOTOPES TO OXIDATION SPECIFIC EPITOPES

(71) Applicants: The Regents of the University of California, Oakland, CA (US); Medizinsche Universitat Wien, Wien (AT)

(72) Inventors: Joseph L. Witztum, San Diego, CA (US); Sotirios Tsimikas, San Diego, CA (US); Christoph Binder, Vienna (AT); Shahzada Amir, Vienna (AT)

(21) Appl. No.: 14/388,739

(22) PCT Filed: Apr. 18, 2013
(86) PCT No.: PCT/US2013/037113

§ 371 Date: Sep. 26, 2014

Related U.S. Application Data
(60) Provisional application No. 61/625,786, filed on Apr. 18, 2012.

(51) Int. Cl. C07K 7/08; G01N 33/68; A61K 39/395; C07K 7/06; C07K 16/18

(52) U.S. Cl. CPC C07K 7/08; C07K 7/06; C07K 16/18; A61K 39/3955; G01N 33/6854; C07K 2317/34; A61K 38/00
USPC 424/139.1; 530/329; 530/328; 530/327; 530/321; 424/185.1; 530/387.9; 436/501

(57) ABSTRACT

Provided herein are peptide mimotopes that are useful for generating antibodies and in the preparation of vaccines and diagnostics for treating and diagnosing coronary artery disease.

### CROSS REFERENCE TO RELATED APPLICATIONS

This application claims priority to U.S. Provisional Application Ser. No. 61/625,786, filed Apr. 18, 2012, the disclosure of which is incorporated herein by reference.

### STATEMENTS REGARDING FEDERALLY SPONSORED RESEARCH

The invention was made with government support under Grant Nos. HL086559 and HL088093 awarded by National Institutes of Health (NIH). The government has certain rights in the invention.

### TECHNICAL FIELD

Compositions and methods for identifying individuals at risk or having cardiovascular disease or disorder.

### BACKGROUND

Atherosclerosis is a chronic inflammatory disease modulated by innate and adaptive immunity. There is emerging interest to identify immunological factors as novel biomarkers for the stratification of cardiovascular risk.

### SUMMARY

Autoantibodies specific for MDA-LDL represent biomarkers to predict cardiovascular risk. However, MDA-LDL is a high variability antigen with limited reproducibility. To identify peptide mimotopes of MDA-LDL, phage display libraries were screened with the MDA-LDL specific IgM monoclonal Ab LRO4, and the specificity and antigenic properties of MDA-mimotopes were assessed in vitro and in vivo. The disclosure provides a 12-mer linear (P1) and a 7-mer cyclic (P2) peptide carrying a consensus sequence, which bound specifically to murine and human anti-MDA monoclonal Abs. Furthermore, MDA-mimotopes were found to mimic MDA-epitopes on the surface of apoptotic cells. Immunization of mice with P2 resulted in the induction of MDA-LDL specific Abs, which strongly immunostained human atherosclerotic lesions. IgG and IgM autoAbs were detected to both MDA-mimotopes in sera of healthy subjects and patients with myocardial infarction and stable angina pectoris undergoing percutaneous coronary intervention, and the titers of autoAbs correlated significantly with respective Ab titers against MDA-LDL.

The disclosure provides specific peptides that are immunological mimotopes of MDA. These mimotopes can serve as standardized and reproducible antigens that will be useful for diagnostic and therapeutic applications in cardiovascular disease.

The disclosure provides a peptide mimotope, wherein the peptide mimotope contains between about 7 and 12 amino acids and specifically binds a polyclonal or monoclonal antibody that is specific for an oxidation-specific epitopes (OSEs) on LDLs. In one embodiment, the peptide mimotope induces the production of antibodies against the peptide mimotope when the peptide mimotope is conjugated to a macromolecular carrier and administered to a subject. In another embodiment, of the foregoing, peptide mimotope specifically binds an MDA-specific murine mAb LRO4 and the human IK17 Fab in a highly specific manner. In another embodiment of any of the foregoing, the peptide mimotope further contains a peptide linker. In yet another embodiment of any of the foregoing, the peptide mimotope is further conjugated to a macromolecular carrier. In a further embodiment, multiple peptide mimotopes are conjugated to a macromolecular carrier. In yet a further embodiment, the peptide mimotope and the macromolecular carrier form a fusion protein. In a further embodiment, the macromolecular carrier is selected from the group consisting of: a tetanus toxoid, a keyhole lympet hemocyanin and an albumin carrier protein. In another embodiment of any of the foregoing, the peptide mimotope is selected from the group consisting of: (a) a linear peptide having a sequence of the general formula X₁X₂X₃X₄X₅X₆X₇X₈X₉X₁₀X₁₁X₁₂ (SEQ ID NO:5), wherein X₁ is an amino acid selected from the group consisting of N, E, Q, A, H, T, G, and D; X₂ is an amino acid selected from the group consisting of S, N, V, R, A, W, Y, and D; X₃ is an amino acid selected from the group consisting of W, R, Y, M, I, L, V, G, T, and P; X₄ is an amino acid selected from the group consisting of T, N, S, and F; X₅ is an amino acid selected from the group consisting of N, K, and S; X₆ is an amino acid selected from the group consisting of A, N, S, D, W, L, Y, T, I, V, K, and P; X₇ is an amino acid selected from the group consisting of S, W, D, T, A, Q, M, E, and P; X₈ is an amino acid selected from the group consisting of L, Q, A, V, G, M, H, S, E, and N; X₉ is an amino acid selected from the group consisting of S, W, H, M, L, A, E, T, D, Q, and R; X₁₀ is an amino acid selected from the group consisting of T, Y, R, S, Q, L, F, V, A, D, and I; X₁₁ is an amino acid selected from the group consisting of F, I, H, L, M V, and P; and X₁₂ is an amino acid selected from the group consisting of H, Q, G, S, M, A, P, W, and L; and (b) a cyclic peptide having a sequence with the general formula X₁X₂X₃X₄X₅X₆X₇ (SEQ ID NO:26), wherein X₁ is selected from the group consisting of N, K, Q, and D; X₂ is selected from the group consisting of N and W; X₃ is selected from the group consisting of W, R, Y, Q, S, and A; X₄ is selected from the group consisting of N, K, H, and P; X₅ is selected from the group consisting of M, Q and H; X₆ is selected from the group consisting of P, R and F; and X₇ is selected from the group consisting of L and T. In yet a further embodiment, the peptide of part (a) comprises a sequence selected from the group consisting of: NSWTNASLSTFH (SEQ ID NO:6), NSRTNNSQWTFQ(SEQ ID NO:7), ESWTNSWAHYFG(SEQ ID NO:8), ESWTNSWAMYFG(SEQ ID NO:9), QSYTNDDVLRIS(SEQ ID NO:10), QNMNNWTLASIM(SEQ ID NO:11), EVMNNWTLASIM(SEQ ID NO:12), ASISNLTLSRFM(SEQ ID NO:13), HSWSNYWGHQHA(SEQ ID NO:14), HRISNYAMELHS(SEQ ID NO:15), HSLTNTQMTQLS(SEQ ID NO:16), HSLSNIQMATLA(SEQ ID NO:17), HRMTNAMHHFMG(SEQ ID NO:18), HRMTNNAMDVFM(SEQ ID NO:19), HRLTNSEQAALP(SEQ ID NO:20), TAVTNSMMERLW(SEQ ID NO:21), GWGNKTPSQDVH(SEQ ID NO:22), DYTNSVSMRYLS(SEQ ID NO:23), HQLSNKDEQTPQ(SEQ ID NO:24), and ADPFSPTNRIPL(SEQ ID NO:25). In a specific embodiment, the peptide of part (a) comprises a sequence HSWTNSWMATFL (SEQ ID NO:1). In yet another embodiment, the peptide of part (b) comprises a sequence selected from the group consisting of NNWNMPL (SEQ ID NO:27); NNRNMPL (SEQ ID NO:28); NNYNMPL (SEQ ID NO:29); NNQNMPL (SEQ ID NO:30); NNWKMPL (SEQ ID NO:31); NNSHMPL (SEQ ID NO:32); KNSXQPL (SEQ ID NO:33); NNSXMPL (SEQ ID NO:34); QNSHMPL (SEQ ID NO:35); NNSNMPL (SEQ ID NO:2); NNSKMRL (SEQ ID NO:36); and DWAPHFT (SEQ ID NO:37). In a specific embodiment, the peptide of part (b) is a cyclic peptide containing a sequence NNSNMPL (SEQ ID NO:2). In another embodiment of aspect of the foregoing the peptide further comprising from 1-10 additional amino acids at either then N-terminal or C-terminal ends of the peptide.

The disclosure also provides composition comprising the peptide mimotope described herein. The composition can comprise the peptide mimotope and a pharmaceutically acceptable carrier.

The disclosure also provides a peptide mimotope of the disclosure conjugated to a carrier protein or adjuvant.

The disclosure also provides a peptide mimotope of the disclosure coated on a substrate.

The disclosure also provides a peptide mimotope of the disclosure further comprising a detectable label.

The disclosure also provides a method of detecting antibodies to oxLDL comprising (a) contacting the substrate coated with a peptide mimotope of the disclosure with a biological sample and detecting the presence of antibodies that bind to the peptide mimotope on the substrate; or (b) contacting a sample with a labeled peptide mimotope of the disclosure and detecting a complex comprising an antibody bound to the labeled peptide; wherein antibodies that bind to the peptide mimotope are indicative of a subject having antibodies to oxLDL adducts.

The disclosure also provides a method of treating or inhibiting atherogenesis in a subject, the method comprising administering to the subject an immunogenic amount of a peptide mimotope of the disclosure or a composition comprising such peptide mimotopes, wherein the administration results in the production of antibodies that bind to oxLDLs. The method can further include administering the peptide mimotope in combination with an immunostimulant adjuvant.

The disclosure also provide an antibody that is produced against a peptide mimotope of the disclosure, wherein the antibody binds to oxidized phospholipids. In one embodiment, the antibody specifically binds to an MDA-derived adduct.

The disclosure also provide a method for ameliorating, reducing or treating the risk of atherosclerosis in a subject, the method comprising administering to the subject antibodies the specifically bind a peptide mimotope of the disclosure in a pharmaceutically acceptable carrier. In one embodiment, the antibody is monoclonal or polyclonal.

The details of one or more embodiments of the disclosure are set forth in the accompanying drawings and the description below. Other features, objects, and advantages will be apparent from the description and drawings, and from the claims.

### BRIEF DESCRIPTION OF THE DRAWINGS

FIGS. 1A-D shows biopanning of phage display peptide libraries with monoclonal antibody LRO4. (A) ELISA for the binding of the murine IgM mAb LRO4 (5 μg/mL) to native LDL (nLDL), CuOx-LDL, MDA-LDL, BSA, and MAA-modified BSA. Values are given as relative light units (RLU) per 100 ms and represent the mean±SD of triplicate determinations. Data are representative of at least three independent experiments. (B) Competition immunoassay for the specificity of LRO4 binding to MDA-LDL. Data are expressed as a ratio of binding in the presence of competitor (B) divided by absence of competitor (B₀) and represent the mean±SD of triplicate determinations. Data are representative of three independent experiments. (C, D) ELISA for the binding of eluted phages to LRO4. After each round of biopanning, the binding of 10¹⁰ pfu/ml phage amplificates from the Ph.D.-12 library (C) and the Ph.D.7C7 library (D) to LRO4 (black bars) and an isotype control IgM (white bars) was tested as described in Methods. Values are given as absorbance measured at 405 and 490 nm and represent the mean±SD of triplicate determinations.

FIGS. 2A-G show that the linear P1 and cyclic P2 peptides mimic a specific MDA-epitope. (A-B) Schematic representation of synthesized peptides carrying the consensus sequence from phages identified with the Ph.D.-12 and Ph.D.C7C library, respectively. (A) P1 is a linear dodecameric peptide (HSWTNSWMATFL) (SEQ ID NO:1) and (B) P2 is a cyclic heptameric peptide (CNNSNMPLC) (SEQ ID NO:2). The peptides' C-terminus containing a GGGC-spacer was amidated. (C, D) ELISA for the binding of LRO4 to P1 and P2. (C) Binding of LRO4 to P1 and P2. Peptides P1, P2, and an irrelevant control peptide were coated at indicated concentrations. Binding of LRO4 (5 μg/mL) was determined by chemiluminescent ELISA. (D) Indicated concentrations of biotinylated peptides were captured on wells pre-coated with 10 μg/mL of neutravidin, and binding of LRO4 (5 μg/mL) was determined as described in Methods. Values are given as RLU per 100 ms and represent the mean±SD of triplicate determinations. (E, F, G) Immunocompetition assays for the antigenic properties of P1 and P2. Binding of 0.5 μg/mL LRO4 to 100 ng/mL captured biotinylated peptides P1 (E) or P2 (F) or to 0.5 μg/mL coated MAA-BSA (G) was measured by ELISA in the presence of increasing concentrations of indicated competitors. Data represent the mean±SD of triplicate determinations and are representative of three independent experiments. Other abbreviations as in legend for FIG. 1.

FIGS. 3A-C show that the mimotopes mimic MDA-epitopes on apoptotic cells. (A) Representative flow cytometry plot of apoptotic Jurkat T-cells. Apoptosis of Jurkat T-cells was induced by 20 mJ/cm² UV irradiation, and cells were stained with PE-labeled Annexin-V and 7-AAD to determine Annexin-V⁻ 7-AAD⁻ viable cells (Q1), Annexin-V⁺ early apoptotic (Q2) and Annexin-V⁺ 7-AAD⁺ late (Q3) apoptotic cells. (B) Representative flow cytometry histogram plot for the binding of apoptotic cells by LRO4. Apoptotic cells were induced as described in (A) and stained with either LRO4, isotype IgM, or no primary Ab, followed by detection using a FITC-conjugated anti-mouse IgM Ab as “secondary Ab” (Sec.Ab). Subsequently, cells were stained with PE-labeled Annexin-V and 7-AAD. The left panel represents staining of viable cells (Q1), the middle panel staining of early apoptotic cells (Q2), and the right panel staining of late apoptotic cells (Q3). (C) Inhibition of LRO4 binding to apoptotic cells by P2. Apoptotic cells were stained with LRO4 in the absence or presence of increasing amounts of P2 or an irrelevant control peptide, as indicated. Data represent the ratio of LRO4 binding as mean fluorescence intensity (MFI) obtained in the presence or absence of competitors (B/B₀). Results are representative of two independent experiments.

FIGS. 4A-C show MDA-mimotope immunization induces MDA-specific Ab responses. (A-B) ELISA for the binding of plasma Abs of immunized mice to MDA-LDL, MAA-LDL, and P2. C57BL/6 mice were immunized with P2-BSA (n=3) or BSA (n=3) as described in Methods. Dilution curves of IgG1 and IgM binding to indicated antigens in pre- and post-immune plasma was determined by chemiluminescent ELISA. (A) Dilution curve of IgG1 binding. (B) Dilution curve of IgM binding. Values are given as relative light units (RLU) per 100 ms and represent the mean±SEM of each group. (C) Immunohistochemical staining of human carotid endarterectomy specimens. Sections were stained with pooled pre-immune (A) or post-immune plasma (B) of P2-BSA immunized mice, or with the MDA-LDL specific mAb MDA2 (C). Positive staining is indicated by red color and nuclei are counterstained with hematoxylin. Other abbreviations are given as in FIG. 1.

FIGS. 5A-F show that the mimotopes are recognized by human autoAbs and mimic MDA-LDL. (A-D) Correlation of mimotope and MDA-LDL specific Ab titers in human plasma. Plasma of middle-aged healthy volunteers (n=18) was obtained in a previous study and Ab titers to P1, P2, and MDA-LDL were measured at 1:400 dilution by chemiluminescent ELISA as described in Methods. (A, C) Correlation of IgM titers to MDA-LDL with IgM titers to (A) P1 and (C) P2. (B, D) Correlation of IgG titer to MDA-LDL with IgG titers to (B) P1 and (D) P2. Values are given as RLU per 100 ms and represent the mean±SD of triplicate determinations. Data points represent measurements of titers obtained from each of the 18 subjects at four different times: 0, 30, 120, and 210 days. Correlations were calculated by analyzing all data from all subjects using non-parametric Spearman's rank correlation (r=spearman rank correlation coefficient). (E, F) Immunocompetition assays for specificity of IgG and IgM Abs to P1. Pooled human plasma was diluted 1:1,000 and incubated with or without increasing concentrations of BSA or MAA-BSA. Subsequently, samples were pelleted and binding of (E) IgM and (F) IgG Abs to coated P1 was determined in supernatants. Data are expressed as B/B₀ and represent the mean±SD of triplicate determinations.

FIGS. 6A-D shows plasma antibody binding to mimotopes over time in patients undergoing percutaneous coronary intervention (PCI). (A-D) ELISA for binding of plasma IgG and IgM to P1 and P2 in patients that underwent PCI. Sequential plasma samples were obtained following PCI. Samples were diluted 1:400 and binding of (A, B) IgM and (C, D) IgG to coated P1 (10 μg/mL; A, C) and P2 (5 μg/mL; B, D) was determined by chemiluminescent ELISA. Shown are relative mean percent changes in Ab binding compared to values obtained at baseline (pre-PCI). *P<0.05, **P<0.01, ***P<0.001.

FIGS. 7A-C shows results of biopanning of M13-phage display (Ph.D.) peptide libraries with LRO4 Abs. (A) A random phage display library is a heterogeneous mixture of phage clones. Each phage carries a DNA fragment of a random peptide fused to the DNA of the outer phage coat protein pIII. Upon expression of the phage coat protein pIII, 5 copies of the random peptide sequence are also displayed. The Ph.D.-12 library consists of random linear peptides with 12 amino acid residues, whereas the Ph.D.-C7C library consists of heptameric peptides flanked by a pair of cysteine residues, which form a disulphide bond, thereby presenting peptides in a cyclic loop. All of the libraries contain a short linker sequence (Gly-Gly-Gly-Ser) between the displayed peptide and outer phage coat protein pIII. The first residue in the peptide is the first randomized amino acid (Ph.D.-12 libraries), whereas in the Ph.D.-C7C library it is preceded by Ala-Cys. (B) For negative selection phages are incubated with isotype control antibodies. In a next step unbound phages are transferred to wells with LRO4 Abs (positive selection). After further washing steps and elution with native LDL, bound phages are eluted with elution buffer or with MDA-LDL and amplified in the E. coli strain ER2738 for application in the next round of biopanning (BPR). (C) After the 3^{rd} BPR, selected single clones are amplified, screened in colony screening assays and by competition ELISA, and subsequently sequenced.

FIGS. 8A-E shows the specificity of LRO4 binding to peptide mimotopes. (A) ELISA for the the binding of LRO4 to BSA, MAA-BSA, the P2 mimotope (ACNNSNMPLC-GGGS) (SEQ ID NO:3) and a scrambled peptide (ACSPNLNMNC-GGGS) (SEQ ID NO:4) of P2 (Scr.P2). Peptides were coated at 20 μg/ml, BSA and MAA-BSA at 5 μg/ml, and binding of indicated concentrations of LRO4 was determined by chemiluminescent ELISA as described in Methods. (B) ELISA for binding of LRO4 and EO6. P1, P2, BSA, MAA-BSA, nLDL, MDA-LDL, and CuOx-LDL were coated at 5-10 μg/mL. Binding of LRO4 and EO6 (both at 5 μg/mL) was determined by chemiluminescent ELISA. (C) Immunocompetition assay for the specificity of LRO4. Binding of 0.5 μg/mL LRO4 to 100 ng/mL captured biotinylated peptides P2 was measured by ELISA in the presence of increasing concentrations of P2 and scrambled P2. Data are given as a ratio of LRO4 binding to P2 in the presence of competitor to the binding in the absence of competitor (B/BJ. (D) ELISA for binding of LRO4 to P2-BSA, BSA, and MAA-BSA, which were coated at 5 μg/mL. Binding of LRO4 (5 μg/mL) was determined by chemiluminescent ELISA. (E) Immunocompetition assays for the specificity of LRO4 binding to P2-BSA. P2-BSA was coated at 5 μg/mL and binding of LRO4 was determined in the absence or presence of soluble BSA, MAA-BSA, P2-BSA, P2, and an irrelevant control peptide at indicated concentrations. Data are expressed as a ratio of binding in the presence of competitor (B) divided by the binding in the absence of competitor (B₀) and represent the mean±SD of triplicate determinations. (A, C, D) Values are given as relative light units (RLU) per 100 ms and represent the mean of duplicate determinations. All data are representative of at least three independent experiments.

FIGS. 9A-D shows that antibodies induced by mimotope immunization stain rabbit atherosclerotic lesions. Immunohistochemical staining of atherosclerotic lesions. Atherosclerotic lesions of WHHL rabbits were obtained as described in Methods and stained with pooled pre-immune (A, C) or post-immune plasma (B, D) of P2-BSA immunized (A, B) or BSA-immunized (C, D) mice. Positive staining is indicated by red color and nuclei are counterstained with hematoxylin.

FIG. 10 show that mimotopes are bound by the human MDA-specific IgG Fab IK17. ELISA for binding of the human monoclonal Fab IK17. P1, P2, and MAA-BSA were coated onto microtiter plates, and biotinylated IK17 (Biot.IK17) was added at indicated concentrations. Binding was detected by chemiluminescent detection using AP-conjugated neutravidin. Values are given as relative light units (RLU) per 100 ms and represent the mean±SD of triplicate determinations.

FIGS. 11A-D shows the dynamics of mimotope-specific Ab titers in plasma of patients after MI. ELISA for binding of IgG and IgM Ab titers to P1 and P2 in plasma of patients suffering an MI (n=7) and of healthy controls (n=18). Plasma was obtained at various time points after MI in a previous study. Samples were diluted 1:400 and binding of (A, B) IgM and (C, D) IgG to coated P1 (10 μg/mL; A, C) and P2 (5 μg/mL; 3, D) was determined by chemiluminescent ELISA as described in Methods. Shown are relative mean percent changes over time in Ab binding compared to values obtained at baseline. MI=myocardial infarctions.

### DETAILED DESCRIPTION

As used herein and in the appended claims, the singular forms “a,” “and,” and “the” include plural referents unless the context clearly dictates otherwise. Thus, for example, reference to “a phospholipid” includes a plurality of such phospholipids and reference to “the protein” includes reference to one or more proteins, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

Any publications discussed above and throughout the text are provided solely for their disclosure prior to the filing date of the present application. Nothing herein is to be construed as an admission that the inventors are not entitled to antedate such disclosure by virtue of prior disclosure.

The disclosure provides methods and compositions useful for treating and diagnosing cardiovascular disease and disorders including coronary artery disease. A “coronary artery disease” (“CAD”) refers to a vascular disorder relating to the blockage of arteries serving the heart. Blockage can occur suddenly, by mechanisms such as plaque rupture or embolization. Blockage can occur progressively, with narrowing of the artery via myointimal hyperplasia and plaque formation. Those clinical signs and symptoms resulting from the blockage of arteries serving the heart are manifestations of coronary artery disease. Atherosclerosis (sometimes called “hardening” or “clogging” of the arteries) is the buildup of cholesterol and fatty deposits (called plaque) on the inner walls of the arteries that restricts blood flow to the heart. Acute Coronary Syndrome is a name given to three types of coronary artery disease that are associated with sudden rupture of plaque inside the coronary artery: unstable angina, Non-ST segment elevation myocardial infarction or heart attack (NSTEMI), or ST segment elevation myocardial infarction or heart attack (STEMI). The length of time that blood flow is blocked and the amount of damage that occurs determines the type of acute coronary syndrome. An acute coronary syndrome can be caused by a small plaque, not necessarily detected by stress testing or cardiac catheterization. Prior symptoms may or may not be present. Manifestations of coronary artery disease include angina, ischemia, myocardial infarction, cardiomyopathy, congestive heart failure, arrhythmias and aneurysm formation. It is understood that fragile plaque disease in the coronary circulation is associated with arterial thrombosis or distal embolization that manifests itself as a myocardial infarction. It is understood that occlusive disease in the coronary circulation is associated with arterial stenosis accompanied by anginal symptoms, a condition commonly treated with pharmacological interventions and with angioplasty.

A “cardiovascular disease” is a cardiovascular disorder, as defined herein, characterized by clinical events including clinical symptoms and clinical signs. Clinical symptoms are those experiences reported by a patient that indicate to the clinician the presence of pathology. Clinical signs are those objective findings on physical or laboratory examination that indicate to the clinician the presence of pathology. “Cardiovascular disease” includes both “coronary artery disease” and “peripheral vascular disease,” both terms being defined below. Clinical symptoms in cardiovascular disease include chest pain, shortness of breath, weakness, fainting spells, alterations in consciousness, extremity pain, paroxysmal nocturnal dyspnea, transient ischemic attacks and other such phenomena experienced by the patient. Clinical signs in cardiovascular disease include such findings as EKG abnormalities, altered peripheral pulses, arterial bruits, abnormal heart sounds, rales and wheezes, jugular venous distention, neurological alterations and other such findings discerned by the clinician. Clinical symptoms and clinical signs can combine in a cardiovascular disease such as a myocardial infarction (MI) or a stroke (also termed a “cerebrovascular accident” or “CVA”), where the patient will report certain phenomena (symptoms) and the clinician will perceive other phenomena (signs) all indicative of an underlying pathology. “Cardiovascular disease” includes those diseases related to the cardiovascular disorders of fragile plaque disorder, occlusive disorder and stenosis. For example, a cardiovascular disease resulting from a fragile plaque disorder, as that term is defined below, can be termed a “fragile plaque disease.” Clinical events associated with fragile plaque disease include those signs and symptoms where the rupture of a fragile plaque with subsequent acute thrombosis or with distal embolization are hallmarks. Examples of fragile plaque disease include certain strokes and myocardial infarctions. As another example, a cardiovascular disease resulting from an occlusive disorder can be termed an “occlusive disease.” Clinical events associated with occlusive disease include those signs and symptoms where the progressive occlusion of an artery affects the amount of circulation that reaches a target tissue. Progressive arterial occlusion may result in progressive ischemia that may ultimately progress to tissue death if the amount of circulation is insufficient to maintain the tissues. Signs and symptoms of occlusive disease include claudication, rest pain, angina, and gangrene, as well as physical and laboratory findings indicative of vessel stenosis and decreased distal perfusion. As yet another example, a cardiovascular disease resulting from restenosis can be termed an in-stent stenosis disease. In-stent stenosis disease includes the signs and symptoms resulting from the progressive blockage of an arterial stent that has been positioned as part of a procedure like a percutaneous transluminal angioplasty, where the presence of the stent is intended to help hold the vessel in its newly expanded configuration. The clinical events that accompany in-stent stenosis disease are those attributable to the restenosis of the reconstructed artery.

A “cardiovascular disorder” refers broadly to both to coronary artery disorders and peripheral arterial disorders. The term “cardiovascular disorder” can apply to any abnormality of an artery, whether structural, histological, biochemical or any other abnormality. This term includes those disorders characterized by fragile plaque (termed herein “fragile plaque disorders”), those disorders characterized by vaso-occlusion (termed herein “occlusive disorders”), and those disorders characterized by restenosis. A “cardiovascular disorder” can occur in an artery primarily, that is, prior to any medical or surgical intervention. Primary cardiovascular disorders include, among others, atherosclerosis, arterial occlusion, aneurysm formation and thrombosis. A “cardiovascular disorder” can occur in an artery secondarily, that is, following a medical or surgical intervention. Secondary cardiovascular disorders include, among others, post-traumatic aneurysm formation, restenosis, and post-operative graft occlusion.

A key problem in treating vascular diseases is proper diagnosis. Often the first sign of the disease is sudden death. For example, approximately half of all individuals who die of coronary artery disease die suddenly, Furthermore, for 40-60% of the patients who are eventually diagnosed as having coronary artery disease, myocardial infarction is the first presentation of the disease. Unfortunately, approximately 40% of those initial events go unnoticed by the patient. The disclosure provides methods and compositions that are useful for determining risk factors and further provide diagnostic agents and methods that can be used to determine whether a subject has coronary artery disease, a cardiovascular disease and/or a cardiovascular disorder.

The methods and compositions of the disclosure can also be used to determine whether a subject has an increased risk of a cardiovascular disease or disorder. “Increased risk” refers to a statistically higher frequency of occurrence of the disease or disorder in an individual in comparison to the frequency of occurrence of the disease or disorder in a population. A factor identified to be associated with increased risk is termed a “risk factor.” An increased level of antibodies that bind to a peptide of the disclosure compared to a normal control is indicative of a risk factor.

A “risk factor” is a factor identified to be associated with an increased risk. A risk factor for a cardiovascular disorder or a cardiovascular disease is any factor identified to be associated with an increased risk of developing those conditions or of worsening those conditions. A risk factor can also be associated with an increased risk of an adverse clinical event or an adverse clinical outcome in a patient with a cardiovascular disorder. Risk factors for cardiovascular disease include smoking, adverse lipid profiles, elevated lipids or cholesterol, diabetes, hypertension, hypercoagulable states, elevated homocysteine levels, increased Lp-PLA₂ and sPLA₂ activity, and lack of exercise.

A mimotope is a macromolecule, often a peptide, which mimics the structure of an epitope. Because of this property it causes an antibody response similar to the one elicited by the epitope. An antibody for a given antigenic epitope will recognize a mimotope which mimics that epitope. Mimotopes are commonly obtained from phage display libraries through biopanning. Vaccines utilizing mimotopes are being developed. In addition, such mimotopes can be used as diagnostic to identify the presence of antibodies to a specific antigen that is “mimicked” by the mimotope.

As used herein the term “mimotope” describes a peptide mimicking an epitope (e.g., a carbohydrate-, peptide, fatty acid-, or a combination thereof, epitope). Mimotopes are useful in diagnostics, therapeutics and vaccines.

Several potential antigens have been found to be present in atherosclerotic lesions of which epitopes of oxidized LDL (OxLDL) appear to be prominent and immunodominant. The oxidative modification of LDL has been shown to result in the generation of various oxidation-specific epitopes (OSEs) that are recognized by specific antibodies (Abs) in a hapten-specific manner. These include adducts with proteins of lipid peroxidation breakdown products such as malondialdehyde (MDA), which forms complex condensation products, as well as the remaining “core aldehydes,” such as 1-palmitoyl-2-(5-oxovaleroyl)-sn-glycero-3-phosphocholine (POVPC). For the assessment of specific immune responses to such OSEs of OxLDL, two model antigens are widely used, namely Cu²⁺-oxidized LDL (CuOx-LDL), which contains many different OSEs, and MDA-modified LDL, which is generated by the derivatization of LDL with various MDA-type adducts. Other exemplary oxidized phospholipids that can be detected using antibodies developed against the mimotopes described herein include oxidized forms of 1-palmitoyl-2-arachidonoyl-sn-glycero-3phos-phorylcholine (Ox-PAPC), 1-palmitoyl-2-oxovaleroyl-sn-glycero-3-phosphoryl-choline (POVPC), 1-palmitoyl-glutaroyl-sn-glycero-3-phosphorylcholine (PGPC), 1-palmitoyl-2-epoxyisoprostane-sn-glycero-3-phosphorylcholine (Ox-SAPC), 1-stearoyl-2-oxovaleroyl-sn-glycero-3-phosphorylcholine (SOVPC), 1-stearoyl-2-glutaroyl-sn-glycero-3-phosphorylcholine (SGPC), 1-stearoyl-2-epoxyisoprostane-sn-glycero-3-phosphorylcholine (SEIPC), 1-stearoyl-2-arachidonyl-sn-glycero-3-phosphorylethanolamine (Ox-SAPE), 1-stearoyl-2-oxovaleroyl-sn-glycero-3-phosphorylethanolamine (SOVPE), 1-stearoyl-2-glutaroyl-sn-glycero-3-phosphorylethanolamine (SGPE), and 1-stearoyl-2-epoxyisoprostane-sn-glycero-3-phosphorylethanolamine (SEIPE).

Antibodies against these two models of OxLDL have been documented in lesions and plasma of patients and animal models of atherosclerosis. In animal studies of atherogenesis, antibody titers to OSEs correlate strongly with the progression and regression of atherosclerosis. In contrast, while many studies in human populations have also shown a positive correlation of such titers with manifestations of cardiovascular disease (CVD), others have not. Furthermore, IgG titers appear to correlate positively, while IgM titers are inversely associated with CVD events. The heterogeneity and complexity of human immunological responses, and the large variation in clinical manifestations studied likely account in part for these differential human results.

The quality and reproducibility of the antigens used may differ substantially between laboratories or even within the same laboratory over time. Indeed, preparation of reproducible CuOx-LDL is difficult, and for this reason, MDA-LDL is commonly used as a model antigen. MDA epitopes are highly expressed in atherosclerotic lesions and that MDA-specific IgM antibodies account for more than 10% of all natural IgM antibodies in mice and represent an equally high percentage of natural IgM in humans. Moreover, in a series of studies immunization with autologous MDA-LDL reduces atherosclerotic lesion formation in rabbits and mice, and the expression of an MDA-specific human monoclonal antibody in cholesterol-fed LDLR^{-/-} mice, by intraperitoneal injection or adenoviral mediated expression, reduces lesion formation by 29-46%. These data suggest that MDA-type epitopes are very prominent, prevalent and important in CVD, and thus represent key candidate antigens to characterize immune responses that are relevant to atherosclerosis. Even here, MDA-modification of LDL results in the formation of various neoepitopes, including the immunodominant malondialdehyde-acetaldehyde (MAA)-type adduct, with different densities and antigenic properties. All of these factors make it difficult to standardize the measurements of autoAb titers to MDA for potential clinical applications, such as biomarkers for CVD, or the use of MDA-LDL as an immunogen in vaccine approaches to atherosclerosis.

Therefore, to develop highly reproducible OSEs that could be used as standard antigens to assess MDA-specific Ab responses, the disclosure provides MDA mimotopes. These mimotopes were identified by screening phage display peptide libraries using an MDA-specific monoclonal Ab. These MDA mimotopes were found to reflect epitopes on MDA-LDL and on apoptotic cells, and when used as immunogens, to induce MDA-specific Ab responses in mice that reacted with epitopes in human atherosclerotic lesions. More importantly, human plasma contained MDA mimotope-specific autoAbs, and that titers to these mimotopes correlated well with titers against the traditional antigen MDA-LDL.

The disclosure provides and characterizes novel peptides mimicking OSE on MDA-LDL. Based on sequences obtained from three rounds of biopanning. These consensus sequences were derived from other sequences set forth in Table 2 (e.g., SEQ ID NOs: 6-37).

The disclosure provides linear mimotopes containing a sequence of the general formula X₁X₂X₃X₄X₅X₆X₇X₈X₉X₁₀X₁₁X₁₂ (SEQ ID NO:5), wherein X₁ is an amino acid selected from the group consisting of N, E, Q, A, H, T, G, and D; X₂ is an amino acid selected from the group consisting of S, N, V, R, A, W, Y, and D; X₃ is an amino acid selected from the group consisting of W, R, Y, M, I, L, V, G, T, and P; X₄ is an amino acid selected from the group consisting of T, N, S, and F; X₅ is an amino acid selected from the group consisting of N, K, and S; X₆ is an amino acid selected from the group consisting of A, N, S, D, W, L, Y, T, I, V, K, and P; X₇ is an amino acid selected from the group consisting of S, W, D, T, A, Q, M, E, and P; X₈ is an amino acid selected from the group consisting of L, Q, A, V, G, M, H, S, E, and N; X₉ is an amino acid selected from the group consisting of S, W, H, M, L, A, E, T, D, Q, and R; X₁₀ is an amino acid selected from the group consisting of T, Y, R, S, Q, L, F, V, A, D, and I; X₁₁ is an amino acid selected from the group consisting of F, I, H, L, M, V, and P; and X₁₂ is an amino acid selected from the group consisting of H, Q, G, S, M, A, P, W, and L. For example, in addition to SEQ ID NO:1 and 3, any of the following mimotopes fit the consensus sequence of SEQ ID NO:5: NSWTNASLSTFH (SEQ ID NO:6), NSRTNNSQWTFQ(SEQ ID NO:7), ESWTNSWAHYFG(SEQ ID NO:8), ESWTNSWAMYFG(SEQ ID NO:9), QSYTNDDVLRIS(SEQ ID NO:10), QNMNNWTLASIM(SEQ ID NO:11), EVMNNWTLASIM(SEQ ID NO:12), ASISNLTLSRFM(SEQ ID NO:13), HSWSNYWGHQHA(SEQ ID NO:14), HRISNYAMELHS(SEQ ID NO:15), HSLTNTQMTQLS(SEQ ID NO:16), HSLSNIQMATLA(SEQ ID NO:17), HRMTNAMHHFMG(SEQ ID NO:18), HRMTNNAMDVFM(SEQ ID NO:19), HRLTNSEQAALP(SEQ ID NO:20), TAVTNSMMERLW(SEQ ID NO:21), GWGNKTPSQDVH(SEQ ID NO:22), DYTNSVSMRYLS(SEQ ID NO:23), HQLSNKDEQTPQ(SEQ ID NO:24), ADPFSPTNRIPL(SEQ ID NO:25).

In another embodiment, the disclosure provides cyclic mimotopes containing a sequence with the general formula X₁X₂X₃X₄X₅X₆X₇ (SEQ ID NO:26), wherein X₁ is selected from the group consisting of N, K, Q, and D; X₂ is selected from the group consisting of N and W; X₃ is selected from the group consisting of W, R, Y, Q, S, and A; X₄ is selected from the group consisting of N, K, H, and P; X₅ is selected from the group consisting of M, Q and H; X₆ is selected from the group consisting of P, R and F; and X₇ is selected from the group consisting of L and T. The sequence of SEQ ID NO:26 can have cysteine residues at the N- and C-terminal ends to facilitate cyclization through sulfhydryl bonds. For example, the following peptide sequences meet the criteria of SEQ ID NO:26: NNWNMPL (SEQ ID NO:27); NNRNMPL (SEQ ID NO:28); NNYNMPL (SEQ ID NO:29); NNQNMPL (SEQ ID NO:30); NNWKMPL (SEQ ID NO:31); NNSHMPL (SEQ ID NO:32); KNSXQPL (SEQ ID NO:33); NNSXMPL (SEQ ID NO:34); QNSHMPL (SEQ ID NO:35); NNSNMPL (SEQ ID NO:2); NNSKMRL (SEQ ID NO:36); and DWAPHFT (SEQ ID NO:37).

Any of the mimotope peptides of the disclosure can be synthesized by commonly used methods such as those that include t-BOC or FMOC protection of alpha-amino groups. Both methods involve stepwise synthesis in which a single amino acid is added at each step starting from the C-terminus of the peptide (See, Coligan, et al., Current Protocols in Immunology, Wiley Interscience, 1991, Unit 9). Peptides of the disclosure can also be synthesized by the well-known solid phase peptide synthesis methods such as those described by Merrifield, J. Am. Chem. Soc., 85:2149, 1962; and Stewart and Young, Solid Phase Peptides Synthesis, Freeman, San Francisco, 1969, pp. 27-62) using a copoly(styrene-divinylbenzene) containing 0.1-1.0 mMol amines/g polymer. On completion of chemical synthesis, the peptides can be deprotected and cleaved from the polymer by treatment with liquid HF-10% anisole for about ¼-1 hours at 0° C. After evaporation of the reagents, the peptides are extracted from the polymer with a 1% acetic acid solution, which is then lyophilized to yield the crude material. The peptides can be purified by such techniques as gel filtration on Sephadex G-15 using 5% acetic acid as a solvent. Lyophilization of appropriate fractions of the column eluate yield homogeneous peptide, which can then be characterized by standard techniques such as amino acid analysis, thin layer chromatography, high performance liquid chromatography, ultraviolet absorption spectroscopy, molar rotation, or measuring solubility. If desired, the peptides can be quantitated by the solid phase Edman degradation.

A peptide of the disclosure comprises a polymer in which the monomers are amino acid residues which are joined together through amide bonds. When the amino acids are alpha-amino acids, either the L-optical isomer or the D-optical isomer can be used. A polypeptide encompasses an amino acid sequence and includes modified sequences such as glycoproteins, retro-inverso polypeptides, D-amino acid modified polypeptides, and the like. A polypeptide includes naturally occurring proteins, as well as those which are recombinantly or synthetically synthesized.

In some embodiments, retro-inverso peptides are used. “Retro-inverso” means an amino-carboxy inversion as well as enantiomeric change in one or more amino acids (i.e., levantory (L) to dextrorotary (D)). A polypeptide of the disclosure encompasses, for example, amino-carboxy inversions of the amino acid sequence, amino-carboxy inversions containing one or more D-amino acids, and non-inverted sequence containing one or more D-amino acids. Retro-inverso peptidomimetics that are stable and retain bioactivity can be devised as described by Brugidou et al. (Biochem. Biophys. Res. Comm. 214(2): 685-693, 1995) and Chorev et al. (Trends Biotechnol. 13(10): 438-445, 1995). The overall structural features of a retro-inverso polypeptide are similar to those of the parent L-polypeptide. The two molecules, however, are roughly mirror images because they share inherently chiral secondary structure elements. Main-chain peptidomimetics based on peptide-bond reversal and inversion of chirality represent important structural alterations for peptides and proteins, and are highly significant for biotechnology. Antigenicity and immunogenicity can be achieved by metabolically stable antigens such as all-D- and retro-inverso-isomers of natural antigenic peptides.

In another embodiment, the peptide is produced by recombinant DNA techniques. For example, an oligonucleotide or polynucleotide encoding a peptide of the disclosure can be expressed from an expression vector and the resulting peptide purified using techniques known in the art (e.g., HPLC or other chromtography techniques). The produced peptides may be substantially purified and used in methods of the disclosure or they may be substantially purified and conjugated to a second molecule of interest. Exemplary molecules include polypeptides or peptide (e.g., such as a growth factor, vaccine or immunogen, antibody and the like); small molecule drugs or nano-, micro-particles. Such nano- and micro-particles are useful in diagnostic assays, drug delivery, and therapeutics. For example, nano- and micro-particles can be used for imaging. In another embodiment, the mimotope peptides of the disclosure may include artificial amino acids.

The disclosure provides a plurality of peptides (SEQ ID NOs:1-37) that mimic epitopes of MDA-LDL. A consensus sequence for dodecameric (P1) and heptameric (P2) phagotopes, respectively, were synthesized (SEQ ID NOs:1 and 2, respectively). Both mimotope peptides were bound by the MDA-specific murine mAb LRO4 and the human IK17 Fab in a highly specific manner. The in vivo relevance of peptide mimotopes was examined by demonstrating their ability to fully compete the binding of LRO4 to the surface of apoptotic cells. Moreover, immunization of mice with P2 mimotopes resulted in the induction of MDA-LDL-specific Ab titers, which specifically recognized epitopes in human carotid atherosclerotic lesions. Several lines of evidence are provided herein documenting the capacity of these peptides to mimic epitopes relevant to atherogenesis. Because the exemplified mimotopes, P1 and P2, are recognized by MDA-specific autoAbs in human sera, they represent for the first time highly reproducible and standardized antigens for the determination of anti-MDA-LDL Abs. Thus, future studies using these mimotopes as antigens provide a reliable assay to evaluate the role of MDA-specific Abs as biomarkers for cardiovascular disease (CVD). Finally, in conjunction with different carriers, the newly identified peptides can also be used as immunogens, for example, to study the mechanistic role of MDA-specific immune responses in immunization studies, and may provide the basis for potential atheroprotective vaccine preparations.

In one embodiment, the disclosure provides antigenic peptides useful for generating antibodies that can bind to MDA-LDL molecules. The antigenic peptides comprise a peptide of any of SEQ ID NOs:1-37 or a consensus sequence thereof.

In another embodiment the peptides of the disclosure (e.g., the peptides of Table 2 (SEQ ID NOs: 6-37) or SEQ ID NO:1 or 2) can be used to immunize a subject, wherein the subject produces antibodies that can specifically bind to a MDA-LDL molecule.

In another embodiment, the mimotopes of the disclosure can be used to determine the presence of antibodies directed against MDA-LDL molecules in a subject. The mimotopes can be labeled or immobilized on a substrate and the binding of an antibody present in a sample from a subject measured using methods known in the art.

The OSE mimotopes of the disclosure have significant potential in biotheranostic applications in humans by providing standardized, chemically defined antigens. As an example of their utility, the ability of P1 (SEQ ID NO:1) and P2 (SEQ ID NO:2) to act as surrogates for MDA-LDL in autoAb assays is demonstrated. The disclosure shows that both sera of healthy subjects and patients with CVD contained autoAbs titers against the peptide mimotopes, which correlated positively with MDA-LDL specific titers. The dynamic changes in these titers to MDA-LDL following MI or PCI could be faithfully reproduced using P1 or P2 as antigens. For example, in response to percutaneous coronary intervention (PCI), there was an immediate drop in Ab titers, consistent with binding to OSE antigens released into the circulation, followed thereafter by long-term rises in Ab titers, consistent with anamnestic responses to OSEs, likely reflecting the extent of injury. These data parallel the previous demonstration of the Ab dynamics against MDA-LDL as antigen.

The disclosure also identifies for the first time peptides that mimic lipid peroxidation derived structures. This is noteworthy, as oxidized lipid derivatives are notoriously unstable structures. MDA-mimotopes are not recognized by the mAb EO6, which has specificity for PC. Thus, the identified peptides are highly specific mimics of MDA epitopes of MDA-LDL, which itself is a complex antigen with different types of MDA-neoantigens. MAA-epitopes have been identified as dominant epitopes of MDA-modifications. Indeed, the newly found mimotopes seem to mimic MAA-epitopes in MDA-LDL, as binding of autoAbs to P1 in pooled plasma of healthy subjects was inhibited by MAA-BSA, indicating that the mimotope-specific autoAbs in human plasma have specificity for MAA. These data suggest that the peptide mimotopes predominantly represent the fluorescent MAA type epitopes that occur following MDA modification of proteins. Thus, the specificity may be useful in studies to a restricted set of “MDA-reactive” antibodies to an immunodominant set of MAA modifications.

One of the major limitations of prior studies in oxidized LDLs is preparing a reproducible MDA-LDL, which is generated by MDA modification of freshly isolated LDL. The newly generated MDA-mimotopes identified herein a reliable surrogate for MDA-LDL, and more specifically, for MAA-type autoantibodies. The availability of these small peptide mimotopes enhances the reproducibility and facilitate the standardization of assays for the determination of such autoAbs. Indeed, the finding that a strong positive correlation was not found in all subjects between titers to MDA-LDL and P1/P2 likely reflects subtle differences in disease relevant autoAbs that are masked by the use of “MDA-LDL” as antigen.

Other applications of OSE mimotopes of the disclosure include their use in molecular imaging and as immunogens in atheroprotective vaccines. In prior studies, OSE in atherosclerotic lesions can be targeted and imaged with human and murine oxidation-specific antibodies carried by MRI nanoparticles. These nanoparticles accumulate within macrophages, allowing visualization of active lesions. In an analogous manner, OSE mimotopes may be bound to similar nanoparticles and used as imaging agents, where they would target scavenger receptors on activated macrophages or other cells displaying binding sites that recognize the mimotopes. This approach may be complementary to using OSE-specific monoclonal antibodies, which would bind to extracellular epitopes, for example, on OxLDL, whereas OSE mimotopes might more specifically image macrophages.

Another use would be as an immunogen in atheroprotective vaccines. The atheroprotective effect of immunization strategies using MDA-LDL in animal models has been described. However, use of MDA-LDL as an antigen in human studies would be impractical. The disclosure demonstrates that immunization with a P2 BSA-conjugated OSE mimotope of the disclosure gave rise to anti-MDA-LDL Abs and these Abs immunostained atherosclerotic lesions, demonstrating its potential as an immunogen for such a vaccine.

In yet another embodiment, the peptides of the disclosure can be used in an assay to identify subjects that have antibodies to oxidized LDL epitopes. Such assays are useful in determining the progression or risk of a subject to a cardiovascular disease or disorder. For example, one can take a biological sample (e.g., a serum sample) at different times and measure the amount of antibody in the sample the specifically interact with a peptide antigen of Table 2 or SEQ ID NO:1 and/or 2. In certain embodiments, the antigen is coated on a substrate, bead or detectably labeled.

In yet another embodiment, the antigenic peptides of the disclosure can be labeled with a detectable label and used in vitro or administered in vivo to determine the location of antibodies or activated macrophages in the body that specifically bind to a OSE mimotope peptide of the disclosure. The detectable label may be a radioactive label, a magnetic label, a chemoluminescent label and the like.

In another embodiment, the disclosure provides an array (e.g., a “biochip” or a “microarray”) that includes immobilized OSE mimotopes of the disclosure that facilitate the detection of a particular molecule or molecules in a biological sample. Other biomolecules that identify the biomarkers (e.g., an antibody that binds an oxLDL) can be included in a custom array for detecting OxLDL. The array can be used to determine the efficacy of a treatment or risk of a cardiovascular disease.

The term “array,” as used herein, generally refers to a predetermined spatial arrangement of binding islands, biomolecules, or spatial arrangements of binding islands or biomolecules. Arrays according to the disclosure that include biomolecules immobilized on a surface may also be referred to as “biomolecule arrays.” Arrays according to the disclosure that comprise surfaces activated, adapted, prepared, or modified to facilitate the binding of biomolecules (e.g., peptides of the disclosure) to the surface may also be referred to as “binding arrays.” Further, the term “array” may be used herein to refer to multiple arrays arranged on a surface, such as would be the case where a surface bore multiple copies of an array. Such surfaces bearing multiple arrays may also be referred to as “multiple arrays” or “repeating arrays.” The use of the term “array” herein may encompass biomolecule arrays, binding arrays, multiple arrays, and any combination thereof; the appropriate meaning will be apparent from context. The biological sample can include fluid or solid samples from any tissue of the body including plasma.

An array of the disclosure comprises a substrate. By “substrate” or “solid support” or other grammatical equivalents, herein is meant any material appropriate for the attachment of biomolecules and is amenable to at least one detection method. As will be appreciated by those in the art, the number of possible substrates is very large. Possible substrates include, but are not limited to, glass and modified or functionalized glass, plastics (including acrylics, polystyrene and copolymers of styrene and other materials, polypropylene, polyethylene, polybutylene, polyurethanes, TEFLON®, etc.), polysaccharides, nylon or nitrocellulose, resins, silica or silica-based materials including silicon and modified silicon, carbon, metals, inorganic glasses, plastics, ceramics, and a variety of other polymers. In addition, as is known the art, the substrate may be coated with any number of materials, including polymers, such as dextrans, acrylamides, gelatins or agarose. Such coatings can facilitate the use of the array with a biological sample derived from serum.

A planar array of the disclosure will generally contain addressable locations (e.g., “pads”, “addresses,” or “micro-locations”) of biomolecules (including OSE mimotopes of the disclosure) in an array format. The size of the array will depend on the composition and end use of the array. Arrays containing from about 2 different biomolecules to many thousands can be made. In some embodiments, the compositions of the disclosure may not be in an array format; that is, for some embodiments, compositions comprising a single biomolecule may be made as well. In addition, in some arrays, multiple substrates may be used, either of different or identical compositions. Thus, for example, large planar arrays may comprise a plurality of smaller substrates.

As an alternative to planar arrays, bead based assays in combination with flow cytometry have been developed to perform multiparametric inimunoassays. In bead based assay systems the biomolecules can be immobilized on addressable microspheres. Each biomolecule for each individual immunoassay is coupled to a distinct type of microsphere (i.e., “microbead”) and the immunoassay reaction takes place on the surface of the microspheres. Dyed microspheres with discrete fluorescence intensities are loaded separately with their appropriate biomolecules. The different bead sets carrying different capture probes can be pooled as necessary to generate custom bead arrays. Bead arrays are then incubated with the sample in a single reaction vessel to perform the immunoassay.

Product formation of the biomarker with their immobilized capture biomolecules (e.g., OSEs mimotopes of the disclosure) can be detected with a fluorescence-based reporter system. Biomarkers can either be labeled directly by a fluorogen or detected by a second fluorescently labeled capture biomolecule. The signal intensities derived from captured biomarkers are measured in a flow cytometer. The flow cytometer first identifies each microsphere by its individual color code. Second the amount of captured biomarkers on each individual bead is measured by the second color fluorescence specific for the bound target. This allows multiplexed quantitation of multiple targets from a single sample within the same experiment. Sensitivity, reliability and accuracy are compared to standard microtiter ELISA procedures. With bead based immunoassay systems serum components can be simultaneously quantified from biological samples. An advantage of bead-based systems is the individual coupling of the capture biomolecule to distinct microspheres.

An array of the disclosure encompasses any means for detecting a biomarker molecule such as, for example, antibodies to OSEs such as MDA-LDL. For example, microarrays can be biochips that provide high-density immobilized arrays of recognition molecules (e.g., mimotopes of the disclosure), where biomarker binding is monitored indirectly (e.g., via fluorescence). In addition, an array can be of a format that involves the capture of proteins by biochemical or intermolecular interaction, coupled with direct detection by mass spectrometry (MS).

Arrays and microarrays that can be used with the methods to detect the biomarkers described herein can be made according to the methods described in U.S. Pat. Nos. 6,329,209; 6,365,418; 6,406,921; 6,475,808; and 6,475,809, and U.S. Pat. Publ. No. 20020049152, which are incorporated herein in their entirety. Arrays, to detect specific selections of sets of biomarkers described herein, can also be made using the methods described in these patents.

Surfaces useful according to the disclosure may be of any desired shape (form) and size. Non-limiting examples of surfaces include chips, continuous surfaces, curved surfaces, flexible surfaces, films, plates, sheets, tubes, and the like. Surfaces preferably have areas ranging from approximately a square micron to approximately 500 cm². The area, length, and width of surfaces according to the disclosure may be varied according to the requirements of the assay to be performed. Considerations may include, for example, ease of handling, limitations of the material(s) of which the surface is formed, requirements of detection systems, requirements of deposition systems (e.g., arrayers), and the like.

In certain embodiments, it is desirable to employ a physical means for separating groups or arrays of binding islands or immobilized biomolecules: such physical separation facilitates exposure of different groups or arrays to different solutions of interest. Therefore, in certain embodiments, arrays are situated within wells of 96, 384, 1536, or 3456 microwell plates. In such embodiments, the bottoms of the wells may serve as surfaces for the formation of arrays, or arrays may be formed on other surfaces and then placed into wells. In certain embodiments, such as where a surface without wells is used, binding islands may be formed or biomolecules may be immobilized on a surface and a gasket having holes spatially arranged so that they correspond to the islands or biomolecules may be placed on the surface. Such a gasket is preferably liquid tight. A gasket may be placed on a surface at any time during the process of making the array and may be removed if separation of groups or arrays is no longer necessary.

Modifications or binding of biomolecules in solution or immobilized on an array may be detected using detection techniques known in the art. Examples of such techniques include immunological techniques such as competitive binding assays and sandwich assays; fluorescence detection using instruments such as confocal scanners, confocal microscopes, or CCD-based systems and techniques such as fluorescence, fluorescence polarization (FP), fluorescence resonant energy transfer (FRET), total internal reflection fluorescence (TIRF), fluorescence correlation spectroscopy (FCS); colorimetric/spectrometric techniques; surface plasmon resonance, by which changes in mass of materials adsorbed at surfaces may be measured; techniques using radioisotopes, including conventional radioisotope binding and scintillation proximity assays so (SPA); mass spectroscopy, such as matrix-assisted laser desorption/ionization mass spectroscopy (MALDI) and MALDI-time of flight (TOF) mass spectroscopy; ellipsometry, which is an optical method of measuring thickness of protein films; quartz crystal microbalance (QCM), a very sensitive method for measuring mass of materials adsorbing to surfaces; scanning probe microscopies, such as AFM and SEM; and techniques such as electrochemical, impedance, acoustic, microwave, and IR/Raman detection. See, e.g., Mere L, et al., “Miniaturized FRET assays and microfluidics: key components for ultra-high-throughput screening,” Drug Discovery Today 4(8):363-369 (1999), and references cited therein; Lakowicz J R, Principles of Fluorescence Spectroscopy, 2nd Edition, Plenum Press (1999).

In another embodiment, a pre-packaged diagnostic kit for determining whether a therapy is effective for treating coronary artery disease or whether an anticoagulant therapy is effective, is provided. The kit may include an array as described above, instructions for using the array, and instructions for calculating the amount of an antibody that binds to mimotopes of the array and determining the amount of MDA-LDL or Ox-LDL specific antibodies in the sample.

Such kits may also include, as non-limiting examples, reagents useful for preparing peptides of the disclosure for immobilization onto binding islands or areas of an array, reagents useful for detecting modifications to immobilized biomolecules, or reagents useful for detecting binding of biomolecules from solutions of interest to immobilized biomolecules, and instructions for use. Likewise, arrays comprising immobilized biomolecules may be included in kits. Such kits may also include, as non-limiting examples, reagents useful for detecting modifications to immobilized biomolecules or for detecting binding of biomolecules from solutions of interest to immobilized biomolecules.

A computer system can be used in the methods of the disclosure to store, calculate and compare ratios and values of antibodies that interact with the OSE mimotopes of the disclosure and correlating those values to antibodies to oxLDL (e.g., MDA-LDL). A processor-based system can include a main memory, preferably random access memory (RAM), and can also include a secondary memory. The secondary memory can include, for example, a hard disk drive and/or a removable storage drive, e.g., a floppy disk drive, a magnetic tape drive, or an optical disk drive. The removable storage drive reads from and/or writes to a removable storage medium. The removable storage medium can be a floppy disk, magnetic tape, optical disk, or the like, which is read by and written to by a removable storage drive. As will be appreciated, the removable storage medium can comprise computer software and/or data.

The computer system can also include a communications interface. Communications interfaces allow software and data to be transferred between the computer system and external devices. Examples of communications interfaces include a modem, a network interface (such as, for example, an Ethernet card), a communications port, a PCMCIA slot and card, and the like. Software and data transferred via a communications interface are in the form of signals, which can be electronic, electromagnetic, optical, or other signals capable of being received by a communications interface. These signals are provided to a communications interface via a channel capable of carrying signals and can be implemented using a wireless medium, wire or cable, fiber optics or other communications medium. Some examples of a channel include a phone line, a cellular phone link, an RF link, a network interface, and other communications channels. In this document, the terms “computer program medium” and “computer usable medium” are used to refer generally to media such as a removable storage device, a disk capable of installation in a disk drive, and signals on a channel. These computer program products are means for providing software or program instructions to a computer system.

Computer programs (also called computer control logic) are stored in main memory and/or secondary memory. Computer programs can also be received via a communications interface. Such computer programs, when executed, enable the computer system to perform the features of the methods discussed herein. In particular, the computer programs, when executed, enable the processor to perform the features of the invention. Accordingly, such computer programs represent controllers of the computer system.

In an embodiment where the elements are implemented using software, the software may be stored in, or transmitted via, a computer program product and loaded into a computer system using a removable storage drive, hard drive, or communications interface. The control logic (software), when executed by the processor, causes the processor to perform the functions of the methods described herein.

In another embodiment, the computer-based methods can be accessed or implemented over the World Wide Web by providing access via a Web Page to the methods of the invention. Accordingly, the Web Page is identified by a Universal Resource Locator (URL). The URL denotes both the server machine and the particular file or page on that machine. In this embodiment, it is envisioned that a consumer or client computer system interacts with a browser to select a particular URL, which in turn causes the browser to send a request for that URL or page to the server identified in the URL. Typically the server responds to the request by retrieving the requested page and transmitting the data for that page back to the requesting client computer system (the client/server interaction is typically performed in accordance with the hypertext transport protocol (“HTTP”)). The selected page is then displayed to the user on the client's display screen. The client may then cause the server containing a computer program of the invention to launch an application to, for example, perform an analysis according to the invention.

An exemplary biochemical test for identifying specific proteins, such as OxPL and apoB, employs a standardized test format, such as the Enzyme Linked Immunosorbent Assay or ELISA test, although the information provided herein may apply to the development of other biochemical or diagnostic tests and is not limited to the development of an ELISA test. Various commercially available ELISA kits are available.

The disclosure demonstrates that immunization with OSE mimotopes of the disclosure induces a strong hapten-specific immune response; e.g. directed against the mimotopes, consisting of a Th2-biased cellular response that leads to a marked induction of IgG antibodies. These induced IgG antibodies not only bind the mimotopes but also bind MAA-LDL and MDA-LDL. Also, these IgM antibodies are atheroprotective in mouse models.

Accordingly, the disclosure also provides antigenic compositions for eliciting an immune response to provide atheroprotective immunity in a subject are provided. In addition, antibodies that bind to such antigens present in the composition are provided. Methods of using the compositions and antibodies to treat, inhibit and diagnose atherosclerosis are also provided. Finally, articles of manufacture that contain a composition or antibody of the disclosure, and instructions for using them, are provided. The antigenic compounds and antibodies provide a basis for developing vaccines to treat atherosclerosis. More particularly, these antigens, which are reproducible and highly characterized, when formulated with an appropriate adjuvant or protein carrier, can be used in vaccines for protection against atherosclerosis.

An “antigen” means a substance, such as a foreign substance, that, when introduced into a subject, can stimulate an immune response. Thus, an antigenic composition is any composition comprising such an antigen, i.e. together with a suitable carrier. The antigenic molecules of the disclosure comprise OSE mimotopes as set forth herein including the peptide having sequences as set forth in SEQ ID NOs:1-37.

As used herein, the term “vaccine” means any compound or preparation of antigens (i.e., OSE mimotopes of the disclosure) desired to stimulate a primary immune response, resulting in proliferation of the memory cells and the ability to exhibit a secondary memory or anamnestic response upon subsequent exposure to the same antigens or recognized epitopes thereof.

The phrase “pharmaceutically acceptable” refers to molecular entities and compositions that are physiologically tolerable and do not typically produce an allergic or similar undesirable reaction, such as gastric upset, dizziness, fever and the like, when administrated to a human. Typically, as used herein, the term “pharmaceutically acceptable” means fulfilling the guidelines and approval criteria of a European Community country's Drug Registration Agency concerning products to be used as a drug, or means that the pharmaceutically acceptable compound, composition, method or use, is listed in the European Community country's Pharmacopoeia or other generally recognized pharmacopoeia for use in animals, and more particularly in humans.

The term “pharmaceutical carrier” refers to a diluent, adjuvant, excipient, or vehicle with which the compound/antigen/antibody is administered. Such pharmaceutical carriers include but are not limited to sterile liquids, such as water and oils, including those of petroleum, oil of animal-, vegetable-, or synthetic origin, such as whale oil, sesame oil, soybean oil, mineral oil and the like. Water or aqueous solutions, saline solutions, and aqueous dextrose and glycerol solutions are typically employed as carriers, particularly for injectable solutions, droplet-dispensed solutions and aerosols.

The term “adjuvant” refers to a compound or mixture that enhances the immune response to an antigen. An adjuvant can serve as a depot that slowly releases the antigen and also as a lymphoid system activator that non-specifically enhances the immune response (Hood et al., Immunology, Second Ed., 1984, Benjamin/Cummings: Menlo Park, Calif., p. 384). Often, a primary challenge with an antigen alone, in the absence of an adjuvant, will fail to elicit a humoral or cellular immune response. Adjuvants include, but are not limited to, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, keyhole limpet hemocyanins, dinitrophenol, and potentially useful human adjuvants such as BCG (bacille Calmette-Guerin) and Corynebacterium parvum. In some instances the adjuvant or carrier can be a serum albumin. Typically, the adjuvant is pharmaceutically acceptable.

The primary purpose of an adjuvant is to enhance the immune response to a particular antigen of interest. In the context of antibody production for research purposes, adjuvants stimulate the rapid and sustained production of high titers of antibodies with high avidity. This permits ready recovery of antibody for further research in vitro. Adjuvants have the capability of influencing titer, response duration, isotype, avidity and some properties of cell-mediated immunity.

Adjuvants may act through three basic mechanisms. The first is to enhance long term release of the antigen by functioning as a depot. Long term exposure to the antigen should increase the length of time the immune system is presented with the antigen for processing as well as the duration of the antibody response. The second is the interaction the adjuvant has with immune cells. Adjuvants may act as non-specific mediators of immune cell function by stimulating or modulating immune cells. Adjuvants may also enhance macrophage phagocytosis after binding the antigen as a particulate (a carrier/vehicle function).

Selection of an adjuvant is based upon antigen characteristics (size, net charge and the presence or absence of polar groups). Adjuvant choice is also dependent upon selection of the species to be immunized. Adjuvant selection remains largely empirical. Antigens that are easily purified or available in large quantities may be good choices for starting with the least inflammatory adjuvants for immunization. Should antibody response not be suitable, a gradual increase in the inflammatory level of the adjuvant would then be warranted. Antigens which are difficult to come by (e.g., very small quantities are available) may be better choices for complexing with the more inflammatory adjuvants such as CFA. In addition, small molecular weight compounds and others known to be weakly immunogenetic, may need to be complexed with CFA to obtain good antibody titers. Exemplary adjuvants include:
Complete Freund's Adjuvant (CFA) is a mineral oil adjuvant that uses a water-in-oil emulsion which is primarily oil. It generally contains paraffin oil, killed mycobacteria and mannide monoosleate. The paraffin oil is generally not metabolized; it is either expressed through the skin (via a granuloma or abscess) or phagocytized by macrophages.
Incomplete Freund's Adjuvant (IFA) is a mineral oil adjuvant with a composition similar to CFA but lacking killed mycobacteria.
Montanide ISA (incomplete seppic adjuvant) is mineral oil adjuvant that uses mannide oleate as the major surfactant component.
Ribi Adjuvant System (RAS) is an oil-in-water emulsion that contains detoxified endotoxin and mycobacterial cell wall components in 2% squalene.
TiterMax is a water-in-oil emulsion that combines a synthetic adjuvant and microparticulate silica with the metabolizable oil squalene. The copolymer is the immunomodulator component of the adjuvant. Antigen is bound to the copolymer and presented to the immune cells in a highly concentrated form.
Syntex Adjuvant Formulation (SAF) is a preformed oil-in-water emulsion that uses a block copolymer for a surfactant. A muramyl dipeptide derivative is the immunostimulatory component. The components are subsequently included in in squalene, a metabolizable oil.
Aluminum Salt Adjuvants are most frequently used as adjuvants for vaccine antigen delivery and are generally weaker adjuvants than emulsion adjuvants.
Nitrocellulose-adsorbed antigen provides the slow degradation of nitrocellulose paper and prolonged release of antigen.
Encapsulated or entrapped antigens permit prolonged release of antigen over time and may also include immunostimulators in preparation for prolonged release.
Immune-stimulating complexes (ISCOMs) are antigen modified saponin/cholesterol micelles. They generally form stable structures that rapidly migrate to draining lymph nodes. Both cell-mediated and humoral immune responses are achieved. Quil A and QS-21 are examples of ISCOMS.
GerbuR is an aqueous phase adjuvant and uses immunostimulators in combination with zinc proline.

Methods of stimulating an immune response in a subject against atherogenesis by administering to the subject an immunogenic amount of an OSE mimotope, wherein the administration results in the production of antibodies that bind to an MDA- and MAA-derived adduct associated with atherosclerosis, are provided.

Anti-atherogenesis compositions containing an immunogenic amount of an OSE mimotope are also provided. Such compositions are useful as vaccines to prevent atherosclerotic lesions from developing. For example, such vaccines can be administered to subjects prior to, contemporaneous with, or subsequent to surgical procedures performed to eliminate occluded blood vessels. The treatment would be useful to prevent or inhibit restenosis. Restenosis is a re-narrowing or blockage of an artery at the same site where treatment, such as an angioplasty or stent procedure, has already taken place. If restenosis occurs within a stent that has been placed in an artery, it is technically called “in-stent restenosis”, the end result being a narrowing in the artery caused by a build-up of substances that may eventually block the flow of blood. Compositions disclosed herein are useful for preventing restenosis by inhibiting the ability of OxLDL to interact with macrophages and promote atherogenesis.

The disclosure further relates to antibodies for the prevention and/or treatment of atherosclerosis. In a first embodiment, an antibody is raised against an OSE mimotope and which is capable of binding not only the mimotope but MAA- and MDA-adducts. Such antibodies are produced by administering the antigenic composition containing one or more OSE mimotopes of the disclosure as a vaccine.

The antibodies according to the disclosure will be administered in one or more dosages, and the amount needed will depend on which phase of the disease the therapy is given as well as on other factors. In order to produce the antibodies, the antigenic composition comprising the OSE mimotopes according to the disclosure will be administered to a subject in order to induce the production of the above described antibodies. Typically, the antibodies will be monoclonal antibodies. Once obtained, such antibodies may be produced by conventional techniques and used in therapy. In general, a monoclonal antibody to an epitope of an antigen can be prepared by using a technique which provides for the production of antibody molecules from continuous cell lines in culture and methods of preparing antibodies are well known to those skilled in this field (see e.g. Coligan (1991) Current Protocols in Immunology, Wiley/Greene, NY; Harlow and Lane (1989) Antibodies: A Laboratory Manual, Cold Spring Harbor Press, NY; and Goding (1986) Monoclonal Antibodies: Principles and Practice (2nd ed) Academic Press, New York, N.Y.). For therapeutic purposes, there may be an interest in using human antibodies.

For therapeutic purposes, the antibody is formulated with conventional pharmaceutically or pharmacologically acceptable vehicles for administration, conveniently by injection. Vehicles include deionized water, saline, phosphate-buffered saline, Ringer's solution, dextrose solution, Hank's solution, etc. Other additives may include additives to provide isotonicity, buffers, preservatives, and the like. The antibody may be administered parenterally, typically intravenously or intramuscularly, as a bolus, intermittently or in a continuous regimen.

In another embodiment, pharmaceutical compositions comprising an OSE mimotope of the disclosure or antibodies directed to such mimotopes are provided. A pharmaceutical composition of the disclosure is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be useful to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

The disclosure further provides an article of manufacture comprising packaging material and, contained within the packaging material, a pharmaceutical composition comprising an immunogenic amount of an OSE mimotope of the disclosure, wherein the packaging material comprises a label or package insert indicating that said composition modulates atherogenesis.

The disclosure encompasses pharmaceutical compositions comprising an antigenic composition or antibody of the disclosure contained in a container and labeled with instructions for use as an atherosclerosis. The pharmaceutical composition can be included in a kit with instructions for use of the composition in the treatment of an atherogenesis-associated disorder. The kit can further comprise instructions for using dosage. Accordingly, the invention contemplates an article of manufacture comprising packaging material and, contained within the packaging material, a composition that modulates atherosclerotic plaque formation and/or persistence. The disclosure further contemplates an article of manufacture comprising packaging material and, contained within the packaging material, a compound that modulates the activity of atherosclerosis.

Methods for treating or ameliorating atherosclerosis in a subject by administering to the subject an OSE mimotope, in a pharmaceutically acceptable carrier, are also provided. In addition, methods for ameliorating atherosclerosis in a subject, by administering to the subject antibodies that bind to an MDA- or MAA-adduct in a pharmaceutically acceptable carrier, are also provided. The disclosure further provides methods of treating or ameliorating a disease caused by atherogenesis in a subject by inducing an immune response in the subject with an OSE mimotope, wherein the subject generates antibodies that bind to the mimotope and also bind epitopes on oxidized lipids (e.g., MDA- and MAA-adducts, and wherein the antibodies prevent the generation of atherogenic plaques, uptake of low density lipoproteins by macrophages, and/or the like thereby ameliorating disease caused by atherogenesis.

### EXAMPLES

### Antigens and Antibodies

Human native LDL, MDA-LDL, MAA-modified LDL, CuOx-LDL, and MAA-BSA were prepared. BSA was purchased from Sigma-Aldrich (St. Louis, Mo., USA). LRO4 is a monoclonal IgM Abs that was cloned from the spleens of cholesterol-fed Ldlr^{-/-} mice, and selected for binding to MDA-LDL using methods known in the art. EO6 is an extensively described phosphocholine (PC)-specific murine IgM mAb that binds to the PC of oxidized phospholipids. MDA2 is a murine IgG monoclonal Ab raised against murine MDA-LDL in BALB/c mice. IK17 is a human IgG Fab fragment specific for an MDA-epitope on MDA-LDL and CuOx-LDL.

Phage Library and Biopanning.

For the identification of peptide mimotopes of MDA-epitopes in MDA-LDL, commercial random phage display peptide libraries purchased from New England Biolabs (NEB) (Beverly, Mass., USA) were screened using the MDA-specific IgM mAb LRO4. Phage display peptide libraries (Ph.D.-C7C & Ph.D.-12) consisted of a linear combinatorial 12-mer (Ph.D.-12) or a cyclic 7-mer peptide flanked by cysteine residues (Ph.D.-C7C) fused to the outer minor phage coat protein (pIII) of M13 phage. Library screening was performed according to NEB's instructions with some modifications (FIG. 71 depicts the steps involved in this procedure). Three rounds of biopanning were performed, in which high specificity was obtained by increasing the concentration of Tween 20 (Sigma-Aldrich) in washing buffers as well as the number of washing steps with each round of biopanning. Furthermore, specificity was increased by decreasing the concentration of coated LRO4 mAb as well as the incubation time of phages with LRO4.

Briefly, LRO4 or a control isotype anti-KLH IgM Ab (C48-6; BD Bioscience-Pharmingen, USA) were coated in buffer containing 0.1 M NaHCO₃ (pH 8.5) on ELISA plates (Nunc Maxisorp, Roskilde, Denmark) overnight (ON) at 4° C. at a concentration of 100 μg/ml for the 1^{st}, 50 μg/ml for the 2^{nd}, and 10 μg/ml for the 3^{rd} biopanning round. The next day, wells were washed with TBS containing 0.05% v/v Tween 20 (TBS-T) and incubated with blocking buffer containing 0.1M NaHCO₃ (pH 8.6) and 5 mg/mL bovine serum albumin (BSA) for 1 hour at room temperature (RT). After further washing with TBS-T, 10 μL of a library solution (containing 2×10¹² phages) diluted in 100 μl of blocking buffer were first added to wells coated with control IgM (negative selection) and then unbound phages were transferred to wells coated with LRO4 (positive selection) and incubated at RT. The incubation time for negative or positive selection was reduced from 60 min for the 1^{st} round to 30 min for the 2^{nd} as well as 3^{rd} biopanning round. Thereafter, plates were washed 5 times with TBS-T to remove unbound phages. To further elute nonspecifically bound phages, wells were incubated with 100 μL of 100 μg/ml of native LDL for 1 hour at RT. After washing up to 5 times with TBS-T, bound phages were eluted with 100 μL of elution buffer containing 0.2 M glycine-HCl (pH 2.2) and 1 mg/mL BSA. Eluates were transferred to microfuge tubes and neutralized with 15 μL of 1 M Tris-HCL (pH 9.1). The eluted phages were then titrated and amplified for the next round of panning. The 2^{nd} and 3^{rd} biopanning round was carried out using amplified eluates from the first and second round as input phages, respectively. To remove nonspecifically bound phages and to increase the affinity in the subsequent biopanning rounds, wells were washed 15 and 30 times with 0.1% TBS-T in the 2^{nd} and 0.5% TBS-T in the 3^{rd} rounds of biopanning, respectively. In the last biopanning round, LRO4-bound phages were competitively eluted with increasing concentrations (3-150 μg/mL) of MDA-LDL diluted in blocking buffer followed by elution using elution buffer.

Plaque Forming Assays and Phage ELISA.

Plaque forming units (pfu) assay was carried out as described in NEB's instructions. The number of LRO4-reactive phages from each elution step was determined by infection of Escherichia coli bacteria (strain 2738, NEB), which were subsequently plated on X-gal/IPTG (Isopropyl β-D-1-thiogalactopyranoside/5-bromo-4-chloro-3-indolyl-beta-D-galactopyranoside) (Sigma-Aldrich) agar plates and resulting plaques containing phages were counted.

Phage ELISA was performed as described by NEB with minor modifications. Ninety six-well ELISA plates (Nunc Maxisorp) were coated with 5 μg/mL LRO4 mAb or control IgM mAb in NaHCO₃ buffer (pH 8.6) at 50 μL/well ON at 4° C. Wells were washed with TBS containing 0.5% Tween 20 and then blocked with blocking buffer (TBS-T containing 1% BSA) at 200 μL/well for 1 hour at RT. After further washing, 10¹⁰ pfu/ml of phage amplificates diluted in blocking buffer were added to the wells at 50 μL/well for 2 hours at RT. Wells were washed again, and an HRP-labeled anti-M13 mAb conjugate (no. 27-9421-01; GE Healthcare, Amersham, UK) diluted 1:1,000 in blocking buffer was added for 1 hour at RT followed by the addition of an 2,2′-azino-bis(3-ethylbenzothiazoline-6-sulphonic acid) (ABTS) (Sigma-Aldrich) substrate solution for 1 hour at RT. The binding reactivity of selected phage clones was measured at OD 405-490 nm using a BioTek Synergy 2 plate reader.

For competitive phage ELISA, plates were coated with 5 μg/mL LRO4 and binding of 25 μL of phage solution at 2×10¹⁰ pfu/mL was tested in the presence of 25 μL of MDA-LDL at 100 μg/mL. Bound phages were detected as described above and data expressed as values obtained in presence of competitor (B) divided by the values obtained in the absence of competitor (B₀). A reciprocal competition assay was performed in which 50 μL of 5 μg/mL MDA-LDL was coated on microtiter wells, and binding of LRO4 that was preincubated for 30 min at RT with a solution containing either no or 1×10¹⁰ pfu/mL phages with or without peptide was tested by chemiluminescent ELISA.

Phage sequencing and peptide synthesis. Single stranded phage DNA from amplified single phage clones was prepared using the Qiaprep spin M13 kit (Qiagen, Hilden Germany). The DNA content was electrophoresed on a 1.2% agarose gel containing 0.01% ethidium bromide in Tris-Borate-EDTA buffer (TBE-buffer) and was visualized by UV illumination. DNA sequencing was performed by VBC Biotech Service GmbH using 96 gIII sequencing primers (NEB) corresponding to the phages' minor coat protein (pIII) gene sequence. Peptide sequences were deduced from DNA sequences. Dodecamer and heptamer peptide sequences were aligned by Clustal W program to obtain consensus sequences. A dodecamer linear peptide P1 (HSWTNSWMATFL; SEQ ID NO:1), a cysteine-constrained heptamer cyclic peptide P2 (AC-NNSNMPL-C; SEQ ID NO:2) and scrambled peptide of P2 (AC-SPNLNMN-C(SEQ ID NO:4)), and a control irrelevant peptide (IMGVGAVGAGAI (SEQ ID NO:38)) were synthesized by Peptide 2.0 Inc. (Chantilly, Va., USA). A spacer (GGGS (SEQ ID NO:39) or GGGC (SEQ ID NO:40) or GGGK (SEQ ID NO:41))-CONH2 was added at each C-terminus. The purity of the all peptides was between 89-95% as assessed by high performance liquid chromatography and mass spectral analysis. For evaluation of its immunogenicity, P2 peptides were conjugated to BSA via the C-terminal cysteine.

Chemiluminescent ELISA.

Binding of mAb as well as plasma Abs to respective antigens was measured by chemiluminescent ELISA. Antigens were coated at 5 μg/mL in PBS/EDTA (pH 7.4). Synthetic peptides were directly coated at 10 μg/mL (P1) or 5 μg/mL (P2) in 0.1M NaHCO₃ buffer (pH 8.6), unless indicated differently. Biotinylated peptides were immobilized at indicated concentrations in 0.1M NaHCO₃ buffer (pH 8.6) on wells pre-coated with 10 μg/mL neutravidin (Pierce, Rockford, Ill., USA). Ab binding was measured using alkaline phosphatase (AP) labeled secondary Abs (described below) followed by chemiluminescent detection. For the detection of human autoAbs, a 1:400 plasma dilution was used. For human assays, internal controls consisting of high and low standard plasma samples were included on each microtiter plate to detect potential variations between microtiter plates. The intra-assay coefficients of variation for all assays were 10 to 14%.

The following secondary Abs were used: Alkaline Phosphatase (AP)-labeled goat anti-mouse IgM (μ-chain specific), IgG (γ-chain specific), IgG1 (Sigma-Aldrich) and IgG2c (Southern Biotech). For the detection of human autoAbs AP-labeled goat anti human IgG (γ-chain specific) and IgM (μ-chain specific) (Sigma-Aldrich) were used. Biotin-conjugated Abs were detected with AP-conjugated neutravidin (1:10,000; Pierce).

Competition Immunoassays.

The specific binding of LRO4 to mimotopes was determined by competition immunoassays. Fifty μl of 0.5 μg/mL MAA-BSA or 100 ng/mL of biotinylated peptides were plated as described above. LRO4 antibody was incubated in TBS-1% BSA with or without increasing concentrations of competitors (BSA, MAA-BSA, P1, P2 and control peptide) for 30 minutes at RT. Binding of LRO4 was determined by chemiluminescent ELISA. The specificity of human plasma Ab binding to P1 was determined in a similar manner. In preliminary experiments, aliquots of pooled plasma samples (n=26) from a published cohort of healthy subjects were diluted in 1% BSA-TBS to yield a limiting plasma dilution. Diluted plasma (1:1,000) and increasing concentrations of competitors were incubated overnight at 4° C. Samples were then centrifuged at 15,800 g for 45 minutes at 4° C. to pellet immune complexes, and supernatants were analyzed for binding to plated P1 (10 μg/mL) by chemiluminescent ELISA.

Immunization Studies.

Two groups (n=3/group) of 5-weeks old male C57BL/6 mice (The Jackson Laboratory, Bar Harbor, Me., USA) were immunized with either a mimotope P2-BSA conjugate or BSA alone. For the primary immunization, 100 μg of antigen in PBS was emulsified in equal volumes of complete Freund's adjuvant (CFA; Sigma-Aldrich) and 50 μL of the homogenized suspension was injected subcutaneously at two sites. For the subsequent 4 booster immunizations every 2-3 weeks afterwards, 100 μg of antigen was emulsified in Incomplete Freund's adjuvant (IFA; Sigma-Aldrich) and 100 μL were injected intraperitoneally. Blood was taken from the tail vein on day 0 (preimmune serum), 21, 35, 47, and 70, and plasma stored at −80° C. for further analyses. The experimental protocol was approved by the Animal Subjects Committee of the University of California, San Diego.

Immunohistochemistry.

Immunostaining of formal sucrose-fixed, paraffin-embedded sections of aortas of atherosclerotic Watanabe heritable hyperlipidemic (WHHL) rabbits and human carotid atherosclerotic endarterectomy lesions was performed. Sections of human or rabbit lesion were blocked with PBS containing 5% horse serum and stained with diluted (1:200 or 1:400) pre- and post-immune sera from P2-BSA and BSA immunized mice, followed by addition of a biotinylated horse anti-mouse IgG (Jackson ImmunoResearch Laboratories, West Grove, Pa., USA). A Vectastain ABC-alkaline phosphatase kit and a Vector Red alkaline phosphatase chromogenic substrate (Vector Labs, Burlingame, Calif., USA) were used to visualize Ab staining. Slides were counterstained with Weigert's Iron Hematoxylin (Richard-Allan Scientific, Kalamazoo, Mich., USA). Immunostaining of adjacent sections in the absence of primary Abs or the MDA-specific mAb MDA2 (5 μg/mL) were used as negative and positive controls, respectively.

Flow Cytometry.

Binding of LRO4 IgM to apoptotic Jurkat T-cells was assessed by flow cytometry. Apoptosis of Jurkat cells was induced by UV irradiation (UV Stratalinker, Stratagene, USA) at 20 mJ/cm², followed by further incubation of the cells in serum free medium for 14-16 hours. After washing in FACS buffer (PBS with 0.1% BSA) 10⁶ cells were incubated with 1 μg LRO4 in 100 μl FACS buffer for 30 minutes at 4° C. In parallel experiments, cells were incubated with LRO4 in the presence of increasing concentrations of P1, P2, and an irrelevant control peptide. Following a washing step, cells were stained with FITC-labeled anti-mouse IgM (II/41, BD Biosciences-Pharmingen) in 100 μL staining buffer for 30 minutes at 4° C. in darkness and washed again. Thereafter, cells were incubated with PE-labeled Annexin-V and 7-AAD (BD Bioscience-Pharmingen) in Annexin-V binding buffer for 15 minutes and immediately analyzed by flow cytometry using a BD LSR II analyzer (BD Bioscience, San Jose, Calif., USA). More than 5.0×10⁴ cells were acquired per sample, and data analysis was performed using FlowJo software (Tree Star Inc, San Carlos, Calif., USA).

Human Subjects.

Human plasma samples from two independent studies were analyzed for the presence of anti-peptide Abs. Details on the study cohorts are described elsewhere and research protocols were approved by the Human Research Protection Program at the University of California, San Diego. For these studies, autoAb titers to MDA-LDL as the antigen have already been reported, to allow ready comparisons to the new data generated here. Study 1 included 18 healthy subjects and 7 patients with acute coronary syndrome treated with primary PCI. Blood was obtained at baseline, and 1, 3, and 7 months thereafter. In addition, plasma from acute coronary syndrome patients was obtained at time of hospital discharge (approximately 4 days after admission). Study 2 included 114 patients with stable angina pectoris undergoing elective PCI. Blood samples were obtained before PCI, immediately after PCI, and 6 and 24 hours, 3 days, 1 week, 1, 3, and 6 months after PCI.

Statistical Analysis.

Statistical analyses were performed with GraphPad Prism 5 for Windows (Graphpad Software, Inc., San Diego, Calif.). Statistical analysis of two groups was performed using the Student's t test. For continuous variables, differences were evaluated by one-way analysis of variances (ANOVA). Spearman correlations were calculated to summarize the association between mimotope and MDA-LDL specific autoAbs. P≦0.05 was considered statistically significant. Data are presented as mean±SD, if not indicated otherwise.

Selection of Phages that Display Peptide Mimotopes of MDA-Epitopes.

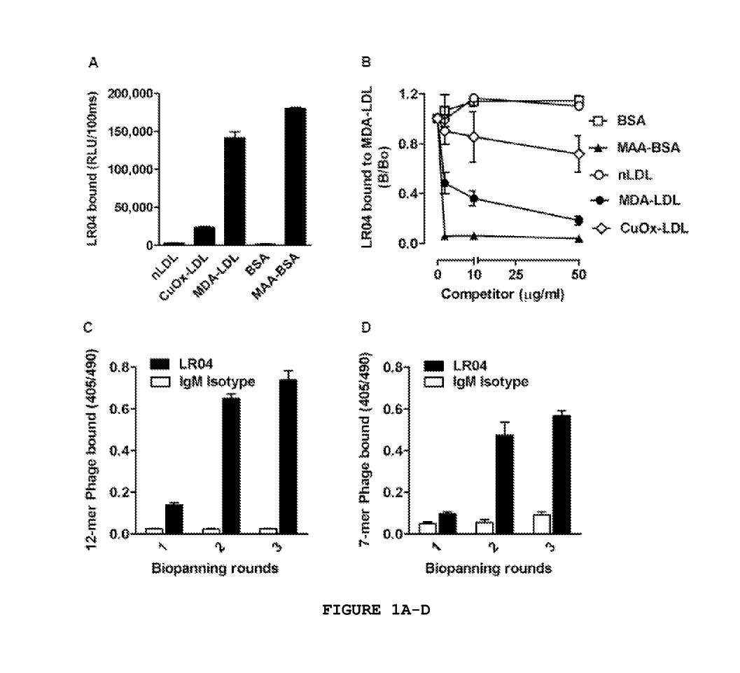

To generate peptide mimotopes of MDA-epitopes present on MDA-LDL, peptide libraries were screened by biopanning for phages reactive with LRO4. LRO4 is a murine IgM mAb that was selected for its ability to bind MDA-LDL. As shown in FIG. 1A, LRO4 binds to MDA-LDL and to a much lesser degree to CuOx-LDL, but not native LDL. Moreover, LRO4 also binds MAA-modified BSA, but not unmodified BSA, demonstrating specificity for advanced MDA-adducts. (FIG. 1A). In immunocompetition assays, the high specificity of LRO4 for MDA-adducts was confirmed, as binding to coated MDA-LDL was efficiently competed by increasing concentrations of soluble MDA-LDL as well as MAA-BSA, but not native LDL or unmodified BSA (FIG. 1B). CuOx-LDL only showed moderate competition, consistent with the presence of minimal amounts of MDA-adducts in these preparations.

Two phage peptide libraries, one displaying 12-mer linear peptides (Ph.D-12) and one displaying cyclic heptamer peptides constrained by flanking cysteines (Ph.D.7C7) were screened for binding to coated LRO4 in three successive biopanning rounds. Enrichment for phages binding to LRO4, but not a control IgM Ab, was confirmed by phage ELISA (FIGS. 1 C-D). A 336- and 400-fold enrichment of phage titers (determined by plaque forming units (pfu) assay) was found after three rounds of selection with the Ph.D.7C7 library and the Ph.D.-12 library, respectively (Table 1A-B).

Table 1A-B. Enrichment of phage titers following each round of biopanning. Titers of eluted phages carrying LRO4-reactive phagotopes from the Ph.D.7C7 library (A) and the Ph.D.-12 library (B) were determined after each round of biopanning by a plaque forming units assay, as described in Methods. Input indicates the number of phages applied to wells coated with LRO4. Output indicates the number of eluted phages. The ratio indicates the fraction of the number of eluted phages after each round of biopanning divided by the number of phages applied at respective round of biopanning. Phage enrichment is calculated as the multiplier of the output/input ratio from one round to the next. PFU=plaque forming units.

TABLE 1A

| | Input | Output | Ratio | Phage |
|---|---|---|---|---|
| Round | (PFU/ml) | (PFU/ml) | (Output/Input) | Enrichment |
| 1 | 2.10¹¹ | 11 × 10⁴ | 0.55 × 10⁻⁶ | |
| 2 | 2.10¹² | 37 × 10⁷ | 18.5 × 10⁻⁵ | 336 |
| 3 | 2.10¹² | 39 × 10⁷ | 19.5 × 10⁻⁵ | 1 |
| | | | | 336 |

TABLE 1B

| | Input | Output | Ratio | Phage |
|---|---|---|---|---|
| Round | (PFU/ml) | (PFU/ml) | (Output/Input) | Enrichment |
| 1 | 2.10¹¹ | 1.9 × 10⁵ | 0.95 × 10⁻⁶ | |
| 2 | 2.10¹¹ | 152 × 10⁵ | 76 × 10⁻⁶ | 80 |
| 3 | 2.10¹¹ | 780 × 10⁵ | 390 × 10⁻⁶ | 5 |
| | | | | 400 |

To identify individual LRO4-binding phages after the third round of biopanning, 132 single clones from the eluted phages of the Ph.D.-12 and 30 clones from the eluted phages of the Ph.D.-C7C libraries were randomly selected and tested for reactivity with LRO4 and a control IgM by ELISA. Forty-two phage clones (25 dodecamers and 17 heptamers) showed significantly higher binding to LRO4 compared to control IgM (Table 2A-B). No binding of control phages (without a peptide insert) to LRO4 and control IgM was observed. To prove the mimicry of selected phagotopes with MDA-epitopes, a competitive phage ELISA was carried out, in which binding of LRO4 to MDA-LDL was tested in the absence and presence of selected phages. The ability of all clones to inhibit binding of LRO4 to MDA-LDL was >75% (Table 2A-B). Control phages did not show any inhibition in this assay. Selected phage clones with the highest binding capacity and specificity were amplified, sequenced, and amino acid sequences were deduced (Table 2A-B).

Table 2A-B. Binding characteristics of MDA-phagotopes. Sequences and binding characteristics of selected phagotopes from (A) the Ph.D.-12 and (B) the Ph.D.7C7 libraries, respectively. Individual phages were selected and amplified after the third round of biopanning, and binding to an isotype control IgM, LRO4, and LRO4 in the presence of 50 μg/mL MDA-LDL was measured by phage ELISA, as described in Methods. Numbers indicate OD values or for inhibition studies the fraction of OD values obtained in the absence and presence of soluble MDA-LDL (B/B₀). Sequences indicate amino acid residues in peptide sequences that were deducted from the DNA sequence of each phagotope. “X” indicates an unidentified amino acid at this position. * for clones with identical sequence representative data of one clone are provided. (SEQ ID NOs:6-37)

TABLE 2A

| | | | Inhibition | |
|---|---|---|---|---|
| | Binding | Binding | of | |
| | of phages | of phages | binding to | |
| Phage | to control | to | LR04 | |
| clones | IgM | LR04 | (B/Bo) | Peptide sequence |
| A40 | 0.04 | 0.89 | 0.10 | NSWTNASLSTFH |
| A34 | 0.11 | 0.82 | 0.11 | NSRTNNSQWTFQ |
| A36, B41 * | 0.03 | 1.16 | 0.14 | ESWTNSWAHYFG |
| M35 | | | | |
| M2 | 0.03 | 1.17 | 0.20 | ESWNTNSWAMYFG |
| M19 | 0.03 | 1.67 | 0.25 | QSYTNDDVLRIS |
| A31 | 0.08 | 0.85 | 0.25 | QNMNNWTLASIM |
| M30 | 0.03 | 1.04 | 0.06 | EVMNNWTLASIM |
| M15 | 0.04 | 1.34 | 0.15 | ASISNLTLSRFM |
| A32 | 0.02 | 0.98 | 0.18 | HSWSNYWGHQHA |
| G4 | 0.04 | 1.45 | 0.10 | HRISNYAMELHS |
| M31 | 0.03 | 1.35 | 0.40 | HSLTNTQMTQLS |
| M10, G8 * | 0.03 | 1.16 | 0.10 | HSLSNIQMATLA |
| A38 | 0.21 | 0.64 | 0.10 | HRMTNAMHHFMG |
| M6, G10 * | 0.03 | 1.56 | 0.20 | HRMTNNAMDVFM |
| M3, M5 * | 0.03 | 1.56 | 0.10 | HRLTNSEQAALP |
| M8 | 0.04 | 1.57 | 0.20 | TAVTNSMMERLW |
| A39 | 0.05 | 1.19 | 0.19 | GWGNKTPSQDVH |
| M36 | 0.01 | 1.47 | 0.07 | DYTNSVSMRYLS |
| A33 | 0.05 | 0.63 | 0.14 | HQLSNKDEQTPQ |
| M7 | 0.03 | 1.37 | 0.10 | ADPFSPTNRIPL |

TABLE 2B

| | Binding | Binding | Inhibition | |
|---|---|---|---|---|
| | of phages | of | of binding | |
| Phage | to control | phages | to LR04 | Peptide |
| clones | IgM | to LR04 | (B/Bo) | sequence |
| Ca1, Ca7* | 0.1 | 0.8 | 0.04 | NNWNMPL |
| Ca59, Cb1* | 0.1 | 0.8 | 0.06 | NNRNMPL |
| Cb589 | 0.1 | 0.8 | 0.03 | NNYNMPL |
| Cb9 | 0.01 | 0.7 | 0.02 | NNQNMPL |
| Cb4 | 0.1 | 0.6 | 0.02 | NNWKMPL |
| Ca9, Cb3* | 0.2 | 0.9 | 0.04 | NNSHMPL |
| Ca8, Ca10 | | | | |
| Ca4 | 0.1 | 0.9 | 0.13 | KNSXQPL |
| Ca6 | 0.0 | 0.7 | 0.04 | NNSXMPL |
| Ca3 | 0.1 | 0.5 | 0.04 | QNSHMPL |
| Cb10 | 0.2 | 0.6 | 0.02 | NNSNMPL |
| Ca2 | 0.2 | 0.8 | 0.12 | NNSKMRL |
| Cb2 | 0.2 | 1.0 | 0.03 | DWAPHFT |

Binding Characteristics of Synthesized MDA-Mimotopes.

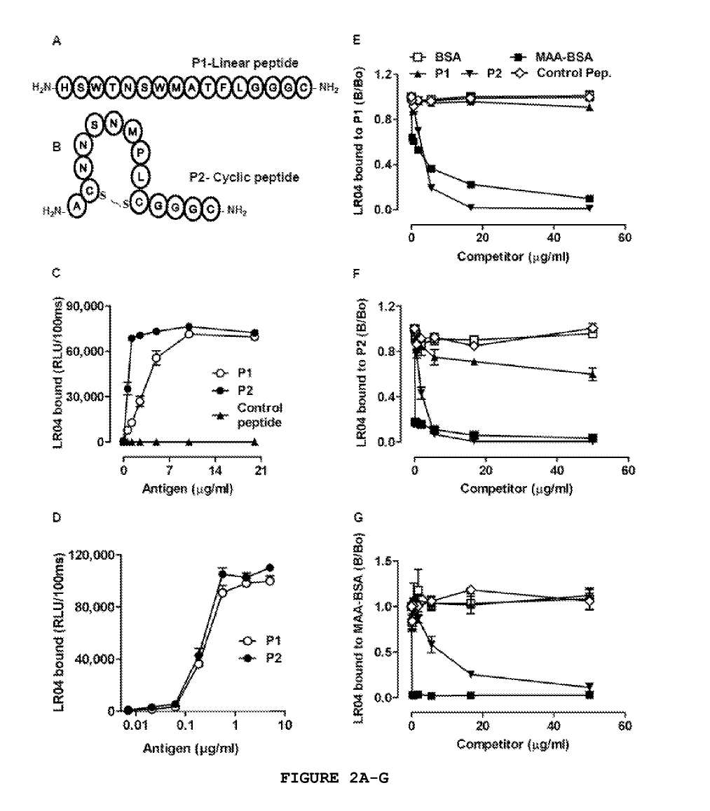

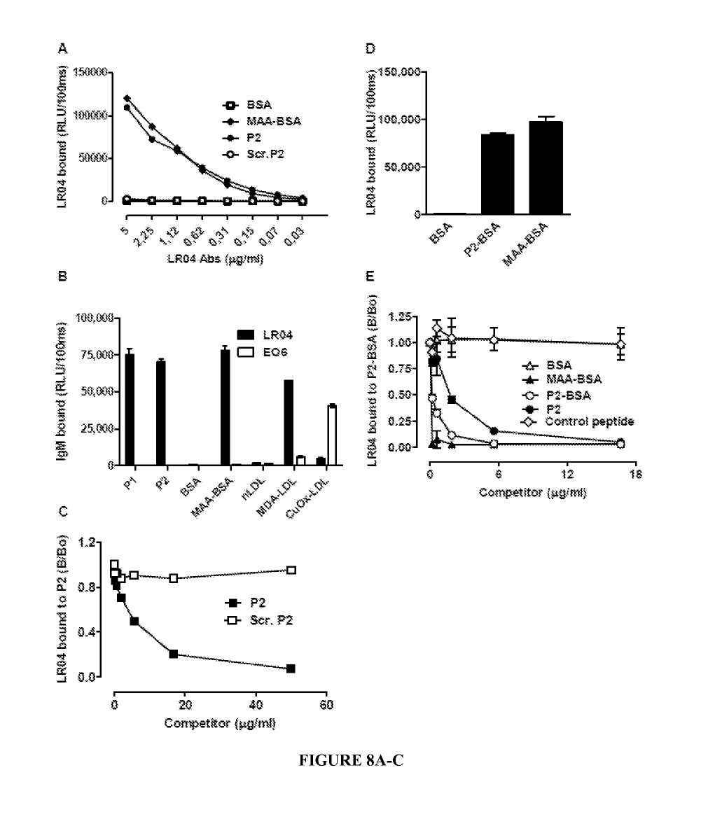

Based on the amino acid sequences deduced, consensus sequences were identified from each library and a dodecameric peptide P1 (HSWTNSWMATFL (SEQ ID NO:1)) (FIG. 2A) and a cyclic heptameric peptide P2 (ACNNSNMPLC (SEQ ID NO:2)) (FIG. 2B) were synthesized. Peptides were directly plated into microtiter wells at increasing concentrations and the binding of LRO4 was tested. Both plated peptides, P1 and P2, but not an irrelevant control peptide (IMGVGAVGAGAI (SEQ ID NO:38)) were bound by LRO4 in a dose dependent manner (FIG. 2C). A scrambled peptide of or scrambled peptide of P2 (ACSPNLNMNC (SEQ ID NO:4)) was not recognized by LRO4 either (FIG. 8A). Binding of LRO4 to the plated peptides was similar to the binding to MDA-LDL and MAA-BSA (FIG. 8B). The PC-specific IgM mAb EO6, which binds CuOx-LDL, did not react with either P1 or P2 (FIG. 8B).

To confirm the binding of LRO4 to the peptides in their native configuration, peptides were designed with a short spacer at the respective C-terminal (GGGK (SEQ ID NO:41)), which was linked to a biotin group. Biotinylated peptides where captured on neutravidin-coated wells and binding of LRO4 to increasing amounts of captured peptides was determined by ELISA. LRO4 recognized both peptides equally well even at concentrations as low as 100 ng/ml (FIG. 2D).

To further test the binding specificity of LRO4 to P1 and P2 and evaluate their function as peptide mimotopes of MDA-epitopes, a series of competition immunoassays were performed. Binding of LRO4 to captured biotinylated P1 (FIG. 2E) and P2 (FIG. 2F) was fully competed by increasing concentrations of soluble MAA-BSA, but not unmodified BSA. Moreover, increasing concentrations of P2 fully competed for LRO4 binding to captured P2 as well as P1. Interestingly, soluble P1 did not compete for LRO4 binding to P1 or P2, respectively (FIGS. 2E-F). Similarly, the irrelevant control peptide and scrambled peptide of P2 had no effect (FIGS. 2E-F and FIG. 8C). In a reciprocal competition experiment, LRO4 binding to coated MAA-BSA was effectively competed by increasing concentrations of MAA-BSA as well as P2, while again, P1 did not have an effect (FIG. 2G). Thus, it appears that when plated, the linear peptide P1 serves as an excellent mimotope for MDA-epitopes, but when in solution loses this property, presumably due to conformational changes. In contrast, the cyclic peptide P2 is an excellent mimotope of MDA-epitopes when plated and in solution.

Peptides Mimic an Epitope Present on Apoptotic Cells.

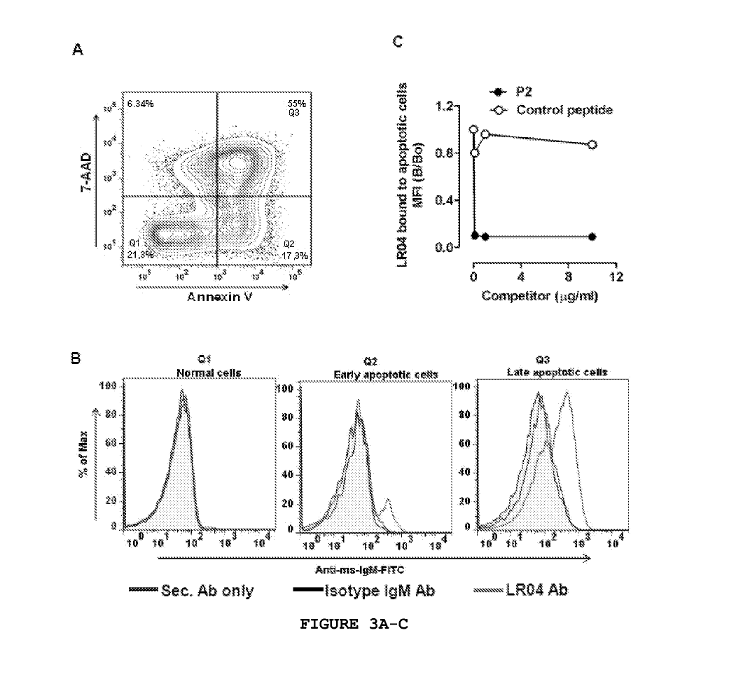

To test whether the peptide mimotopes truly mimic epitopes occurring in vivo, the ability of P2 to inhibit the binding of LRO4 to apoptotic cells was tested. MDA-epitopes are generated when cells undergo apoptosis, and that MDA-epitopes are a major OSE on the surface of apoptotic cells. Consistent with these reports, LRO4 binds to the surface of early and late apoptotic cells, as indicated by Annexin-V and 7-AAD staining (FIGS. 3A-B). Importantly, in the presence of increasing concentrations of soluble P2, but not of an irrelevant control peptide, binding of LRO4 to apoptotic cells was fully inhibited (FIG. 3C). These data demonstrate that the peptide mimotope of MDA, P2, mimics epitopes on the surface of dying cells.

Mimotope Immunization Induces Antibodies Against MDA-LDL.

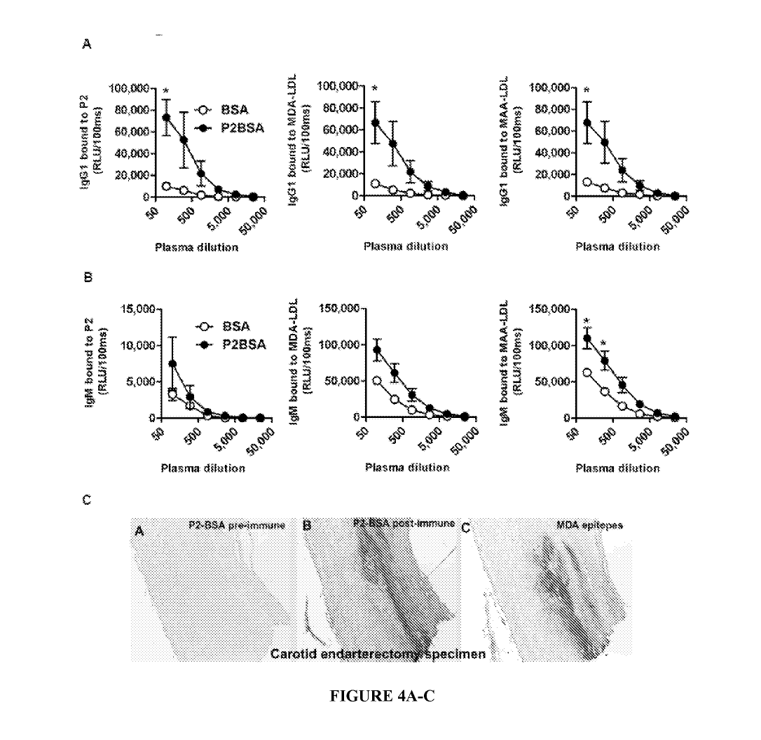

Based on the preceding characterizations, the cyclic P2 peptide was used to test for its immunogenic properties. P2 was conjugated to BSA (P2-BSA) as a carrier protein, which did not affect its reactivity and specificity for LRO4 (FIGS. 8D-E). C57BL/6 mice were immunized with P2-BSA or BSA alone emulsified in Freund's adjuvant, and Ab titers were determined in the plasma obtained after the last boost. Mice immunized with P2-BSA developed high IgG1 titers to BSA and P2 whereas mice immunized with BSA developed only Ab titers to BSA (FIG. 4A). Importantly, mice immunized with P2-BSA displayed a robust IgG1 response to MDA-LDL and MAA-LDL, which was not observed in control mice immunized with BSA alone (FIG. 4A), and a more modest IgG2c response. In addition, the immunization with P2-BSA also induced IgM titers to MDA-LDL and more prominently to MAA-LDL (FIG. 4B). Thus, immunization of mice with P2 mimotopes results in the induction of MDA-specific Abs, similar to that observed following immunization with homologous MDA-LDL.

Antisera from Mimotope Immunized Mice Recognize Epitopes in Atherosclerotic Lesion.

Human carotid endarerectomy specimens and lesions of WHHL rabbits were stained with sera from immunized mice. Post-immune IgG of P2-BSA immunized mice showed high reactivity in both human (FIG. 4C, panel B) and rabbit atherosclerotic lesions (FIG. 9B), similar to that achieved with the MDA-specific monoclonal Ab MDA2, (FIG. 4C, panel C). In contrast, post-immune IgG of BSA immunized mice failed to stain (FIG. 9D). These data indicate that the Abs induced by mimotope immunization are specific for epitopes that occur in atherosclerotic lesions in vivo.

Mimotopes are Recognized by Human Antibodies.

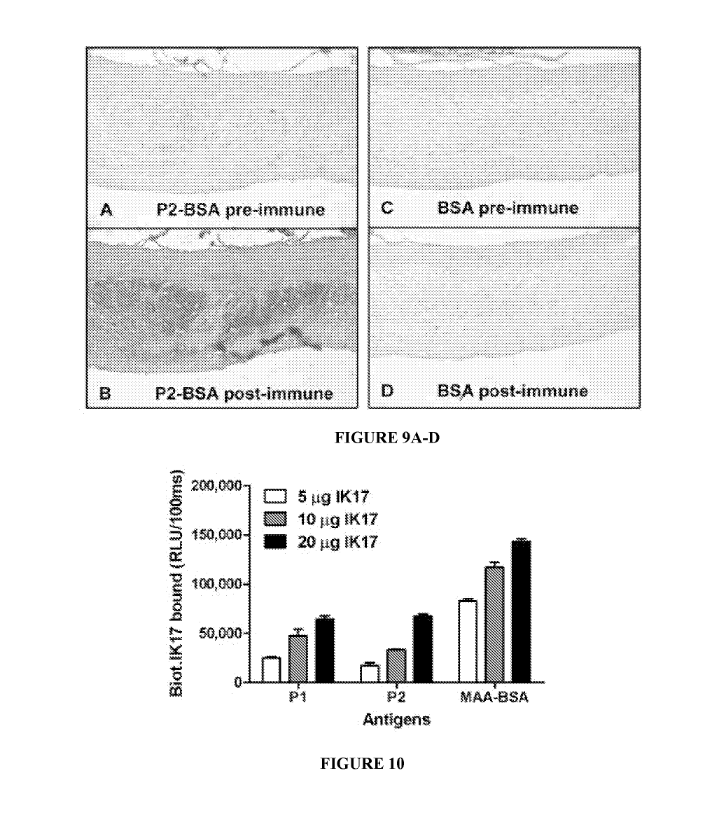

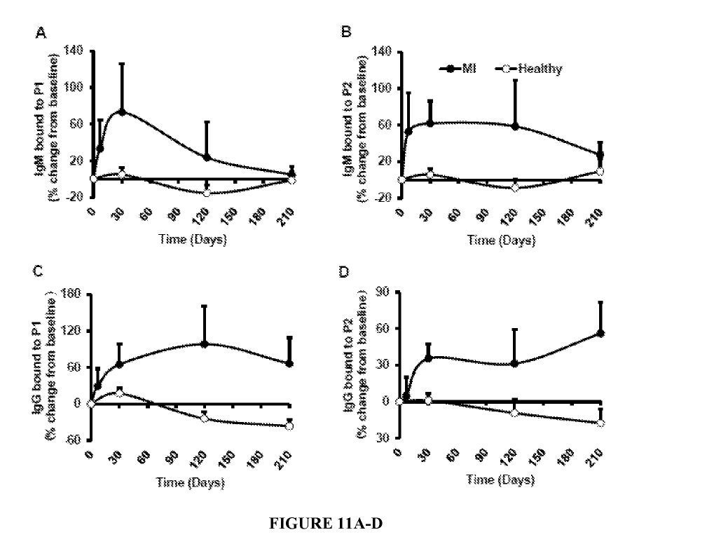

The presence of autoAbs to MDA-LDL in humans in various clinical settings has been demonstrated to be hapten-specific, i.e. recognized MDA epitopes. Given the highly specific nature of P1 and P2 as MDA-mimotopes in mice, it was asked if the newly identified mimotopes would be equally recognized by human autoAbs, as was MDA-LDL. The first test was whether the human MDA-LDL specific monoclonal Ab IK17, which was generated by Ab phage display, also binds the synthetic peptide mimotopes. Indeed, IK17 bound to MAA-BSA and P1 and P2 in a dose-dependent manner (FIG. 10), suggesting that both P1 and P2 could serve as MDA-mimotopes for human autoAbs.

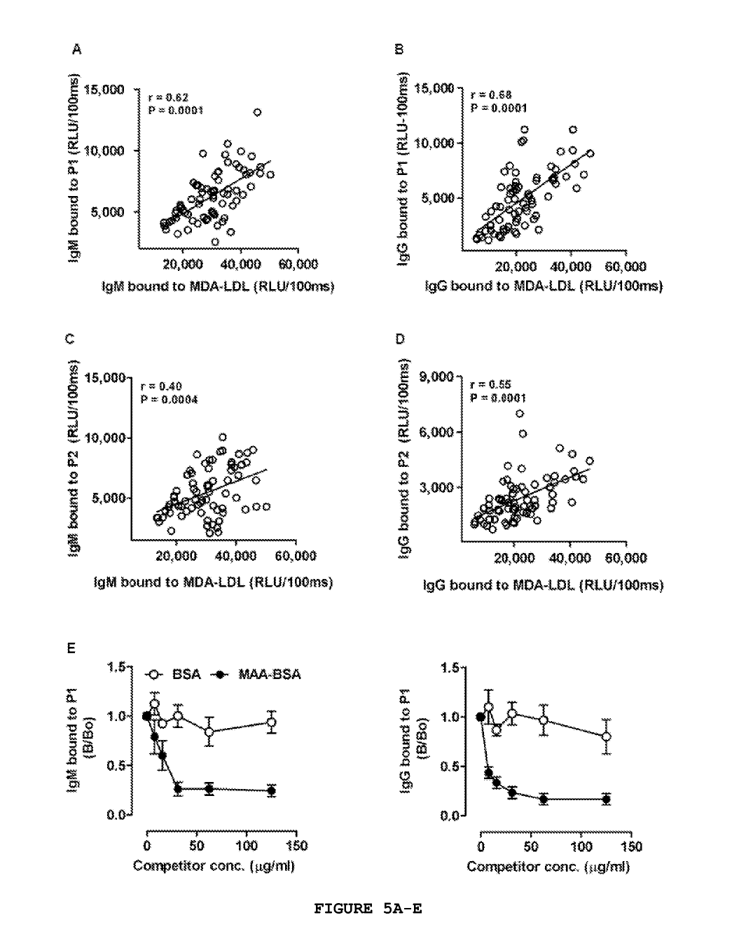

To directly validate this, plasma from a previous study of middle-aged healthy volunteers (n=18) sampled at 4 different time points over 210 days, and measured Ab binding to P1 and P2 as well as to MDA-LDL in parallel assays. Plasma of all individuals contained both IgG and IgM titers to P1 and P2, respectively (FIGS. 5 A-D). Moreover, the Ab titers to MDA-LDL in each plasma sample paralleled the titers to P1 and P2, resulting in significant correlations of IgG and IgM titers to P1 and P2 vs. titers to MDA-LDL (FIG. 5). The specificity of P1-reactive Abs were further studied for MDA-adducts in immunocompetition assays, showing that the binding of both IgM and IgG Abs to plated P1 was nearly completely inhibited by increasing concentrations of soluble MAA-BSA (FIGS. 5 E and F).

Disease-associated changes in MDA-specific Ab titers over a 7-month period using P1 and P2 as antigens were studied. The dynamics of OxLDL-specific Ab titers were studied over time in patients with MI, and found an initial 30% and 50% increase over baseline in anti-OxLDL IgG and IgM titers, respectively. When these same plasma samples were tested for Ab binding to P1 and P2, an even greater increase in both IgG and IgM titers was found to P1 and to a lesser degree to P2 (FIG. 11). While IgM titers to P1 and P2 returned towards baseline after 210 days, IgG titers to both mimotopes remained increased even after 7 months.

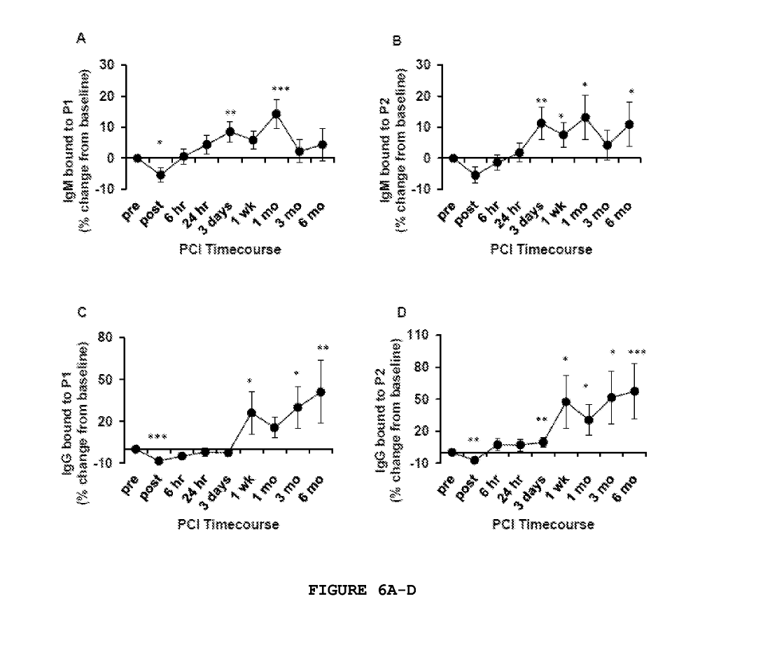

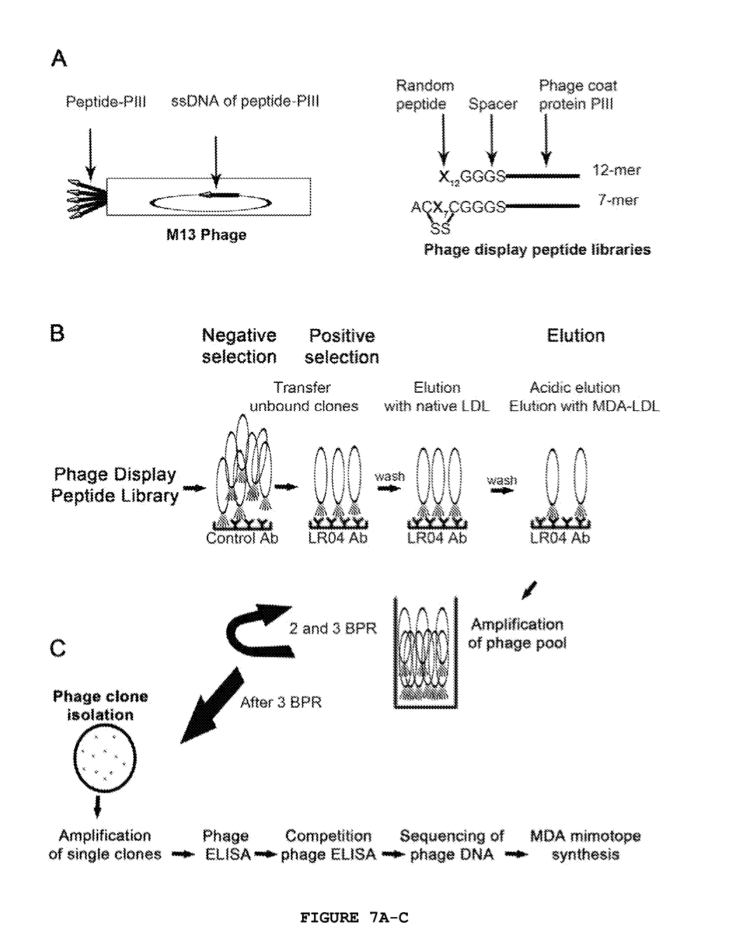

In another study, autoAb titers in plasma from patients with stable angina collected immediately before and serially up to 6 months after PCI were studied. Immediately after PCI, a significant drop in both IgG and IgM titers was found to MDA-LDL that was paralleled by an increase in immune complexes with apoB containing lipoproteins. By 6 hours, these values had returned to baseline, and over the next few weeks there was a rise in both IgG and IgM titers that persisted for up to 6 months. Remarkably, utilizing the P1 and P2 mimotopes as antigens, these patterns of changes were reproduced in both IgG and IgM Ab responses (FIGS. 6 A-D). Anti-P1 IgM and IgG autoAbs positively correlated with previously determined anti-MDA-LDL titers (r=0.75, p<0.0001 and r=0.39, p<0.0001 respectively), as did anti-P2 (r=0.56, p<0.0001 r=0.29, p<0.0001 respectively).

Taken together, these data demonstrate that mimotope-specific Ab titers behave similar to MDA-LDL specific titers following an acute MI or PCI, and they suggest that as a consequence of MI or PCI, antigens are released that trigger an adaptive immune response, which cross-reacts with the newly identified peptide mimotopes. These findings demonstrate that P1 and P2 are mimotopes for relevant MDA antigens.

A number of embodiments of the invention have been described. Nevertheless, it will be understood that various modifications may be made without departing from the spirit and scope of the invention. Accordingly, other embodiments are within the scope of the following claims.

1. A peptide mimotope, wherein the peptide mimotope contains between about 7 and 12 amino acids and specifically binds a polyclonal or monoclonal antibody that is specific for an oxidation-specific epitopes (OSEs) on LDLs.

2. The peptide mimotope of claim 1, wherein the peptide mimotope induces the production of antibodies against the peptide mimotope when the peptide mimotope is conjugated to a macromolecular carrier and administered to a subject.

3. The peptide mimotope of claim 1, wherein the peptide mimotope specifically binds an MDA-specific murine mAb LRO4 and the human IK17 Fab in a highly specific manner.

4. The peptide mimotope of claim 1, wherein the peptide mimotope further contains a peptide linker.

5. The peptide mimotope of claim 1 further conjugated to a macromolecular carrier.

6. The peptide mimotope of claim 5, wherein multiple peptide mimotopes are conjugated to a macromolecular carrier.

7. The peptide mimotope of claim 5, wherein the peptide mimotope and the macromolecular carrier form a fusion protein.

8. The peptide mimotope of claim 5, wherein the macromolecular carrier is selected from the group consisting of: a tetanus toxoid, a keyhole lympet hemocyanin and an albumin carrier protein.

9. The peptide mimotope of claim 1, wherein the peptide mimotope is selected from the group consisting of:
(a) a linear peptide having a sequence of the general formula X₁X₂X₃X₄X₆X₆X₇X₈X₆X₁₀X₁₁X₁₂ (SEQ ID NO:5), wherein X₁ is an amino acid selected from the group consisting of N, E, Q, A, H, T, G, and D; X₂ is an amino acid selected from the group consisting of S, N, V, R, A, W, Y, and D; X₃ is an amino acid selected from the group consisting of W, R, Y, M, I, L, V, G, T, and P; X₄ is an amino acid selected from the group consisting of T, N, S, and F; X₅ is an amino acid selected from the group consisting of N, K, and S; X₆ is an amino acid selected from the group consisting of A, N, S, D, W, L, Y, T, I, V, K, and P; X₇ is an amino acid selected from the group consisting of S, W, D, T, A, Q, M, E, and P; X₈ is an amino acid selected from the group consisting of L, Q, A, V, G, M, H, S, E, and N; X₉ is an amino acid selected from the group consisting of S, W, H, M, L, A, E, T, D, Q, and R; X₁₀ is an amino acid selected from the group consisting of T, Y, R, S, Q, L, F, V, A, D, and I; X₁₁ is an amino acid selected from the group consisting of F, I, H, L, M V, and P; and X₁₂ is an amino acid selected from the group consisting of H, Q, G, S, M, A, P, W, and L; and
(b) a cyclic peptide having a sequence with the general formula X₁X₂X₃X₄X₆X₆X₇ (SEQ ID NO:26), wherein Xi is selected from the group consisting of N, K, Q, and D; X₂ is selected from the group consisting of N and W; X₃ is selected from the group consisting of W, R, Y, Q, S, and A; X₄ is selected from the group consisting of N, K, H, and P; X₅ is selected from the group consisting of M, Q and H; X₆ is selected from the group consisting of P, R and F; and X₇ is selected from the group consisting of L and T.

10. The peptide mimotope of claim 9, wherein the peptide of part (a) comprises a sequence selected from the group consisting of: NSWTNASLSTFH (SEQ ID NO:6), NSRTNNSQWTFQ(SEQ ID NO:7), ESWTNSWAHYFG(SEQ ID NO:8), ESWTNSWAMYFG(SEQ ID NO:9), QSYTNDDVLRIS(SEQ ID NO:10), QNMNNWTLASIM(SEQ ID NO:11), EVMNNWTLASIM(SEQ ID NO:12), ASISNLTLSRFM(SEQ ID NO:13), HSWSNYWGHQHA(SEQ ID NO:14), HRISNYAMELHS(SEQ ID NO:15), HSLTNTQMTQLS(SEQ ID NO:16), HSLSNIQMATLA(SEQ ID NO:17), HRMTNAMHHFMG(SEQ ID NO:18), HRMTNNAMDVFM(SEQ ID NO:19), HRLTNSEQAALP(SEQ ID NO:20), TAVTNSMMERLW(SEQ ID NO:21), GWGNKTPSQDVH(SEQ ID NO:22), DYTNSVSMRYLS(SEQ ID NO:23), HQLSNKDEQTPQ(SEQ ID NO:24), and ADPFSPTNRIPL(SEQ ID NO:25).

11. The peptide mimotope of claim 9, wherein the peptide of part (a) comprises a sequence HSWTNSWMATFL (SEQ ID NO:1).

12. The peptide mimotope of claim 9, wherein the peptide of part (b) comprises a sequence selected from the group consisting of NNWNMPL (SEQ ID NO:27); NNRNMPL (SEQ ID NO:28); NNYNMPL (SEQ ID NO:29); NNQNMPL (SEQ ID NO:30); NNWKMPL (SEQ ID NO:31); NNSHMPL (SEQ ID NO:32); KNSXQPL (SEQ ID NO:33); NNSXMPL (SEQ ID NO:34); QNSHMPL (SEQ ID NO:35); NNSNMPL (SEQ ID NO:2); NNSKMRL (SEQ ID NO:36); and DWAPHFT (SEQ ID NO:37).

13. The peptide mimotope of claim 12, wherein the peptide of part (b) is a cyclic peptide containing a sequence NNSNMPL (SEQ ID NO:2).

14. The peptide mimotope of claim 9, wherein the peptide further comprising from 1-10 additional amino acids at either then N-terminal or C-terminal ends.

15. A composition comprising the peptide mimotope of claim 1.

16. A peptide mimotope of claim 9 conjugated to a carrier protein or adjuvant.

17. A peptide mimotope of claim 1 coated on a substrate.

18. A peptide mimotope of claim 1 further comprising a detectable label.

19. A method of detecting antibodies to oxLDL comprising
(a) contacting the substrate of claim 17 with a biological sample and detecting the presence of antibodies that bind to the peptide on the substrate; or
(b) contacting a sample with a labeled peptide of claim 18 and detecting a complex comprising an antibody bound to the labeled peptide;
wherein antibodies that bind to the peptide bind to oxLDL adducts.

20. A method of treating or inhibiting atherogenesis in a subject, the method comprising administering to the subject an immunogenic amount of a peptide mimotope of claim 1, wherein the administration results in the production of antibodies that bind to oxLDLs.

21. The method of claim 20, wherein the peptide mimotope is administered in combination with an immunostimulant adjuvant.

22. An antibody that is produced against a peptide of claim 9 and wherein the antibody binds to oxidized phospholipids.

23. (canceled)

24. A method for ameliorating atherosclerosis in a subject, the method comprising administering to the subject antibodies that specifically bind a peptide mimotope of claim 1 in a pharmaceutically acceptable carrier.

25. The method of claim 24, wherein the antibody is monoclonal or polyclonal.
